(19) United States
(12) Patent Application Publication
(10) Pub. No.: US 2026/0178028 A1
(43) Pub. Date: Jun. 25, 2026

(54) EXECUTING A COLLABORATION SESSION FOR HUMAN TELEOPERATION OF A ROBOTIC DEVICE PARTICIPANT

(71) Applicant: MICROSOFT TECHNOLOGY LICENSING, LLC, Redmond, WA (US)

(72) Inventor: Daniel ROSENSTEIN, Issaquah, WA (US)

(21) Appl. No.: 18/991,470

(22) Filed: Dec. 21, 2024

(51) Int. Cl. G05D 1/224; G05D 1/223; G05D 101/10

(52) U.S. Cl. CPC G05D 1/2244; G05D 1/223; G05D 2101/10

(57) ABSTRACT

A system implements techniques for executing a collaboration session between at least one human participant, at least one robotic device participant, and at least one artificial intelligence agent participant. The collaboration session can be executed in relation to a mission to be completed within a geographical environment. A mission defines one or more goals. Accordingly, a mission typically includes a set of tasks to be completed to achieve the goals. The system is configured to cause a control element for teleoperating the robotic device participant to be activated (e.g., displayed) in the context of the collaboration session. The control element receives a control input from the human participant. Based on the control input, the system transmits, via the collaboration session, a teleoperation instruction to the robotic device participant.

### BACKGROUND

The use of robotic devices is becoming more prevalent in the world. For instance, different types of robotic devices have recently been configured to perform various tasks for humans. In some cases, the performance of tasks by robotic devices replaces the need for humans to perform the tasks (e.g., dangerous tasks, time-consuming tasks). Thus, any many areas of life, robotic devices have been proven to improve the way in which people live.

The tasks that can be performed by robotic devices are becoming more complex. Furthermore, the tasks that can be performed by robotic devices are becoming interrelated. Unfortunately, existing systems fail to provide a way for effective and efficient coordination of robotic devices that are expected to perform complex and interrelated tasks. It is with respect to these and other considerations that the disclosure made herein is presented.

### SUMMARY

The system described herein implements techniques for executing a collaboration session between at least one human participant, at least one robotic device participant, and at least one artificial intelligence agent participant. A robotic device is a programmable device configured to implement a series of physical actions automatically. In this context, “automatically” means the physical actions are implemented via the embedded programming of the robotic device and/or via remote control of the robotic device (e.g., in a scenario where a human and robotic device are not co-located).

In various examples described herein, the collaboration session is executed in relation to a mission to be completed within a geographical environment. A mission defines one or more goals. Accordingly, a mission typically includes a set of tasks to be completed to achieve the goals. In various examples, the mission is related to a response to an event and the set of tasks to be completed in accordance with the mission is distributed across available resources. More specifically, different human and/or robotic device roles may have varied responsibilities in implementing different tasks to complete the mission.

As an illustrative example, an event may be a natural disaster such as a fire from a lightning strike, a hurricane, or an earthquake. The use of robotic devices can be helpful in responding to a natural disaster event. Typically, multiple organizations respond to execute and/or complete a mission with the goal of helping people that are affected by the natural disaster event. For instance, first tasks for the mission may be related to finding and rescuing survivors (e.g., removing people from dangerous areas). Second tasks for the mission may be related to limiting further damage caused by the natural disaster event (e.g., finding areas where the fire is burning and extinguishing the fire, securing unstable buildings). Third tasks for the mission may be related to identifying damaged/offline “utility” infrastructure (e.g., electric grid infrastructure, water supply infrastructure, gas pipeline infrastructure) and fixing the damage/offline utility infrastructure so it comes back online in due time.

In this illustrative example, the multiple organizations often include different government agencies from local, state, and/or federal jurisdictions. Moreover, the multiple organizations may include private and/or charitable organizations as well. Each of the organizations may include their own personnel, their own experiences and/or procedures with respect to deploying robotic devices, as well as their own execution and/or communications infrastructure to operate the robotic devices. When different personnel and different types of robotic devices from different organizations converge on a geographical environment in response to an event such as a natural disaster, it is difficult to coordinate the tasks so that the mission can be achieved in a more effective and efficient manner.

The collaboration session described herein creates an effective and efficient solution for humans and robotic devices to coordinate the performance of the mission. Additionally, the collaboration session described herein allows for artificial intelligence agents to assist in the coordination of the performance of the mission. For instance, via the execution of a collaboration session, humans from different organizations that use heterogenous robotic devices (e.g., different types of robotic devices, different types of communications) can quickly connect through a central system to collaborate and coordinate performance of tasks that are intended to complete a mission. Moreover, access to an intelligence layer provided by artificial intelligence agents that are able to participate in the collaboration session enhances the collaboration and coordination.

The illustrative example of a natural disaster event provided above (and discussed herein) is a larger-scale event. However, it is understood in the context of this disclosure that a mission can be implemented at different scales. For instance, a mission can also be implemented in response to a smaller-scale event that only requires coordination and collaboration between one human, one robotic device, and one artificial intelligence agent. For example, a person may create a collaboration session to coordinate with a robotic device and/or an artificial intelligence agent to find a particular team member (e.g., determine a current location of the particular team member) in an office building so the team member can resolve a project issue a team has encountered. In this example, the event is the project issue and the mission is finding the particular team member.

As shown via the examples described above, the coordination enabled via the collaboration session described herein may be scaled to apply in any context in which work (e.g., a mission) needs to be done by a human, a robotic device, and an artificial intelligence agent. Various contexts include disaster response, safety and security, healthcare and medical instrumentation, manufacturing and industrial lines/warehouses, office and/or personal management, agriculture, and so forth. Accordingly, a geographical environment, as described herein, can include an identifiable “real-world” setting and/or area. The identifiable real-world setting and/or area can be indoor, such as an office building, a retail building, a personal residence, a warehouse, a hospital, a medical office, a factory floor, a manufacturing line, or other types of settings and/or areas within physical structures that can be blueprint-or human-defined. Alternatively, the identifiable real-world setting and/or area can be outdoor, such as a forest, a mountain, a construction site, a neighborhood, a town, a city, a county, a state, a country, a field, a pasture, or other type of outdoor settings and/or areas that can be map-or human-defined.

Robotic devices can operate on land, on water, in the air, in space, or a combination thereof, and can be programmed to perform different tasks. For example, an unmanned aerial vehicle (UAV) may be tasked with capturing video and/or dropping items from the sky. A sea drone may be tasked with capturing video and/or providing supplies to an area that cannot be reached by land. A bomb disposal robotic device may be tasked with capturing video and/or safely disabling an explosive device. A backhoe robotic device may be tasked with capturing video and/or moving dirt, rocks, and/or rubble. A dump truck robotic device may be tasked with capturing video and hauling away dirt, rocks, and/or rubble. An office or retail robotic device may be tasked with stocking retail and/or supply shelves. A warehouse robotic device may be tasked with sorting items in bins. A manufacturing robotic device may be tasked with connecting two parts of an apparatus. These example robotic devices are just a few of the numerous different types of robotic devices that have been manufactured and configured to perform various tasks in varying contexts.

Regardless of the size and/or scope of the mission and/or a scale of an event to which the mission responds, the collaboration session described herein enables at least one human and one robotic device to work together in conjunction with an artificial intelligence agent. The artificial intelligence agent functions as a translation and/or orchestration interface between the human and the robotic device. The collaboration session presents a low barrier of entry for humans and/or robotic devices to be part of a coordinated mission. Moreover, the collaboration session enables the integration of heterogenous robotic devices (e.g., different fleets of robotic devices) that are not designed and/or configured to communicate with one another. Moreover, through the use of the aforementioned accessible artificial intelligence agent, the collaboration session enables effective participation for humans without detailed working knowledge of the robotic devices deployed to the geographical environment in which the mission is being implemented, thereby reducing the cognitive load required for successful missions and increasing the overall efficiency for mission completion.

The humans, robotic devices, and/or artificial intelligence agents participating in a collaboration session are respectively referred to herein as human participants, robotic device participants, and artificial intelligence agent participants. The disclosed system is configured to expose an application programming interface that allows robotic devices to access and download a “robot agent” that enables robotic device participation in the collaboration session. The robot agent includes centralized code that configures the robotic devices with communication and/or configuration software that is compatible with the collaboration session. That is, after downloading the robot agent, a robotic device can participate in the collaboration session via the communication (e.g., transmission) of robot data.

In one example, the robot data includes sensor data sensed by a sensor embedded in a robotic device participant. More specifically, the sensor data can include one or more of image data (e.g., still images) captured by an image capture device embedded in or attached to the robotic device participant, video data (e.g., a sequence of video frames) captured by a video capture device embedded in or attached to the robotic device participant, audio data captured by a microphone embedded in or attached to the robotic device participant, temperature data captured by a thermometer embedded in or attached to the robotic device participant, air quality data captured by an air quality sensor embedded in or attached to the robotic device participant, pressure data captured by a pressure sensor embedded in or attached to the robotic device participant, velocity data captured by a velocity sensor embedded in or attached to the robotic device participant, smoke data captured by a smoke detecting sensor embedded in or attached to the robotic device participant, gas data captured by a gas detecting sensor embedded in or attached to the robotic device participant, thermal data captured by a thermal sensor embedded in or attached to the robotic device participant, depth data captured by a depth sensor embedded in or attached to the robotic device participant, odor (smell) data captured by an odor sensor embedded in or attached to the robotic device participant, lidar data captured by a laser component embedded in or attached to the robotic device participant, radar data captured by a radar component embedded in or attached to the robotic device participant, or infrared (IR) data captured by an IR sensor embedded in or attached to the robotic device participant. While a list of example types of data and/or sensors is provided above, it is understood in the context of this disclosure, that a robotic device participant can be configured with hardware, firmware, and/or software to detect and/or sense any type of environmental data. In another example, the robot data includes location data for the robotic device (e.g., a Global Positioning System (GPS) location).

The robot agent made available by the system via the application programming interface configures a bi-directional communication bridge between a robotic device and the collaboration session. More specifically, this bi-directional communication bridge connects the robotic device to cloud infrastructure that hosts the collaboration session via different types of networks including private and/or public local area networks (LANs), private and/or public metropolitan area networks (MANs), private and/or public wide area networks (WANs), Wi-Fi networks, public and/or private mobile networks (e.g., 5G networks, LTE networks), satellite networks, radio networks, and so forth.

The collaboration session is started when any of the participants (e.g., a human participant, a robotic device participant, or an artificial intelligence agent participant) creates the collaboration session and joins the collaboration session. The participant that starts the collaboration session can then add other participants to the collaboration session via an invitation to join. In various examples, the invitation to join is a notification that wakes a robotic device participant from a sleep state and/or activates the robotic device agent to enable the bi-directional communication bridge to/from the collaboration session. As described above, after a robotic device participant has joined the collaboration session, the robotic device can start communicating (e.g., reporting) sensor data and/or location data to the collaboration session.

After the collaboration session is started, the system generates an interaction environment for the collaboration session. As described in further detail below, the interaction environment includes a graphical representation for each of a plurality of participants that have joined the collaboration session. As described in further detail below, the graphical representation for more prominent participants can be displayed in an area of the interaction environment that is designated as a primary area. Alternatively, the graphical representation for less prominent participants can be displayed in an area of the interaction environment that is designated as a secondary area. Additionally, the interaction environment includes a selectable element that enables the human participant to switch between at least two viewing states associated with the participants in the collaboration session. The system provides the interaction environment to a computing device associated with the human participant, as further discussed below in the examples of the Detailed Description. Moreover, the system provides a context of the whole interaction environment, or a particular aspect of the interaction environment (e.g., a video stream), to an artificial intelligence agent for processing and analysis.

In further examples described herein, the system is configured to execute, within the interaction environment, a mechanism for a human participant to provide input that describes a task that is part of the mission and that causes an artificial intelligence agent participant to assist with completion of the task that is part of the mission. The system receives, via the mechanism, the input from the human participant. The artificial intelligence agent participant is configured to assist with completion of the task that is part of the mission by generating an instruction for the robotic device participant. The system then transmits, via the collaboration session and based on the input received, the instruction from the artificial intelligence agent participant to the robotic device participant.

In even further examples described herein, the system is configured to execute, within the interaction environment, a mechanism for a human participant to provide input that activates a control element for teleoperating a robotic device participant that has been deployed to the geographical environment to assist with completion of the mission. The system receives, via the mechanism, the input from the human participant. The input causes the control element for teleoperating the robotic device participant to be activated in the interaction environment. In one specific example, activating the control element comprises displaying the control element in the context of the interaction environment. In another specific example, activating the control element configures the interaction environment to engage with teleoperation hardware associated with a computing device being used by the human participant. The system ultimately receives, via the control element, a control input from the human participant and transmits, via the collaboration session and based on the control input received, a teleoperation instruction to the robotic device participant.

This Summary is provided to introduce a selection of concepts in a simplified form that are further described blow in the Detailed Description. This Summary is not intended to identify key or essential features of the claimed subject matter, nor is it intended to be used as an aid in determining the scope of the claimed subject matter. The term “techniques,” for instance, may refer to system(s), method(s), computer-readable instructions, module(s), algorithms, hardware logic, and/or operation(s) as permitted by the context described above and throughout the document.

### BRIEF DESCRIPTION OF DRAWINGS

The Detailed Description is described with reference to the accompanying figures. In the description detailed herein, references are made to the accompanying drawings that form a part hereof, and that show, by way of illustration, specific embodiments or examples. The drawings herein are not drawn to scale. Like numerals represent like elements throughout the several figures.

FIG. 1 illustrates an example environment in which a system executes a collaboration session between at least one human participant, at least one robotic device participant, and at least one artificial intelligence agent participant.

FIG. 2A illustrates an example interaction environment where graphical representation for at least one human participant, at least one robotic device participant, and at least one artificial intelligence agent participant are displayed.

FIG. 2B illustrates another example interaction environment where graphical representations for at least one human participant, at least one robotic device participant, and at least one artificial intelligence agent participant are displayed.

FIG. 3 illustrates further aspects of the system executing a collaboration session.

FIG. 4A illustrates an example interaction environment that shows a human participant providing input to access an options menu.

FIG. 4B illustrates an example interaction environment that shows a human participant providing input to switch a view state with respect to layout and/or participants displayed in areas that are designated as primary.

FIG. 4C illustrates an example interaction environment that has been switched to a different view state based on the human input in FIGS. 4A-4B.

FIG. 4D illustrates an example interaction environment that has been switched to a different view state based on the human input in FIGS. 4A-4B.

FIG. 5A illustrates an example interaction environment that shows a human participant providing input to switch to a view state that includes a map of the geographical environment.

FIG. 5B illustrates an example interaction environment that has been switched to a the view state that includes the map of the geographical environment based on the human input in FIG. 5A.

FIG. 5C illustrates an example interaction environment that shows a human participant providing input to the map to switch a view state with respect to the sensor data being communicated by a robotic device participant.

FIG. 5D illustrates an example interaction environment that has been switched to a different view state based on the human input in FIG. 5C.

FIG. 5E illustrates an example interaction environment that shows additional types of robot data that can be displayed in the interaction environment.

FIG. 6 is a flowchart depicting an example process for executing a collaboration session between at least one human participant, at least one robotic device participant, and at least one artificial intelligence agent participant.

FIG. 7 illustrates further aspects of the system executing a collaboration session, directed to enabling a human participant to provide input that causes an artificial intelligence agent participant to perform an analysis and/or to generate and transmit an instruction to a robotic device participant.

FIG. 8 illustrates further aspects of the system executing a collaboration session in FIG. 7, where autonomous control of a robotic device participant is enabled for an artificial intelligence agent participant based on contextual data associated with a mission and/or a geographical environment in which the mission is being completed.

FIG. 9A illustrates an example interaction environment that exposes a mechanism for a human participant to provide input (e.g., a prompt, a chat message) that causes an artificial intelligence agent participant to perform an analysis on an aspect of the interaction environment.

FIG. 9B illustrates an example of how the analysis by the artificial intelligence agent participant can be displayed.

FIG. 10A illustrates an example interaction environment that exposes a mechanism for a human participant to provide input (e.g., a prompt, a chat message) that causes an artificial intelligence agent participant to generate an instruction for a robotic device participant to execute a task associated with a mission in a geographical environment.

FIG. 10B illustrates an example interaction environment that exposes a mechanism for a human participant to provide a voice-based input that causes an artificial intelligence agent participant to generate an instruction for a robotic device participant to execute a task associated with a mission in a geographical environment

FIG. 10C illustrates an example of how the status of the task being executed by the robotic device participant by can be displayed in the interaction environment.

FIG. 10D illustrates another example of how the status of the task being executed by the robotic device participant by can be displayed in the interaction environment and how autonomous control of the robotic device participant can be implemented by the artificial intelligence agent participant.

FIG. 11 illustrates another example context for an interaction environment where graphical representations for at least one human participant, at least one robotic device participant, and at least one artificial intelligence agent participant are displayed.

FIG. 12 is a flowchart depicting an example process for executing a collaboration session that enables a human participant to provide input that causes an artificial intelligence agent participant to generate and transmit an instruction to a robotic device participant.

FIG. 13 illustrates further aspects of the system executing a collaboration session, directed to enabling a human participant to provide input that causes the collaboration session to activate (e.g., display) a control element for a robotic device participant and/or to transmit a teleoperation instruction to the robotic device participant in response to control input received via the control element.

FIG. 14A illustrates an example interaction environment that exposes a mechanism for a human participant to provide input that causes the collaboration session to activate (e.g., display) a control element for a robotic device participant.

FIG. 14B illustrates an example interaction environment that displays a universal control element configured to receive control input that causes a common teleoperation instruction to be transmitted to different types of robotic device participants.

FIG. 14C illustrates an example interaction environment that displays a universal control element configured to receive control input that causes a customized teleoperation instruction to be transmitted to a specific type of robotic device participant.

FIG. 14D illustrates an example interaction environment that displays a customized control element configured to receive control input that causes a customized teleoperation instruction to be transmitted to a specific type of robotic device participant.

FIG. 15 illustrates an example piece of teleoperation hardware that is a sensory form factor device associated with a glove.

FIG. 16 is a flowchart depicting an example process for executing a collaboration session that enables a human participant to provide input that causes the collaboration session to activate (e.g., display) a control element for a robotic device participant and/or to transmit a teleoperation instruction to the robotic device participant in response to control input received via the control element.

FIG. 17 is a computer architecture diagram illustrating an illustrative computer hardware and software architecture for a computing system capable of implementing aspects of the techniques and technologies presented herein.

### DETAILED DESCRIPTION

The system described herein implements techniques for executing a collaboration session between at least one human participant, at least one robotic device participant, and at least one artificial intelligence agent participant. In various examples described herein, the collaboration session is executed in relation to a mission to be completed within a geographical environment. A mission defines one or more goals. Accordingly, a mission typically includes a set of tasks to be completed to achieve the goals. In various examples, the mission is related to a response to an event and the set of tasks to be completed in accordance with the mission is distributed across available resources. More specifically, different human and/or robotic device roles may have varied responsibilities in implementing different tasks to complete the mission.

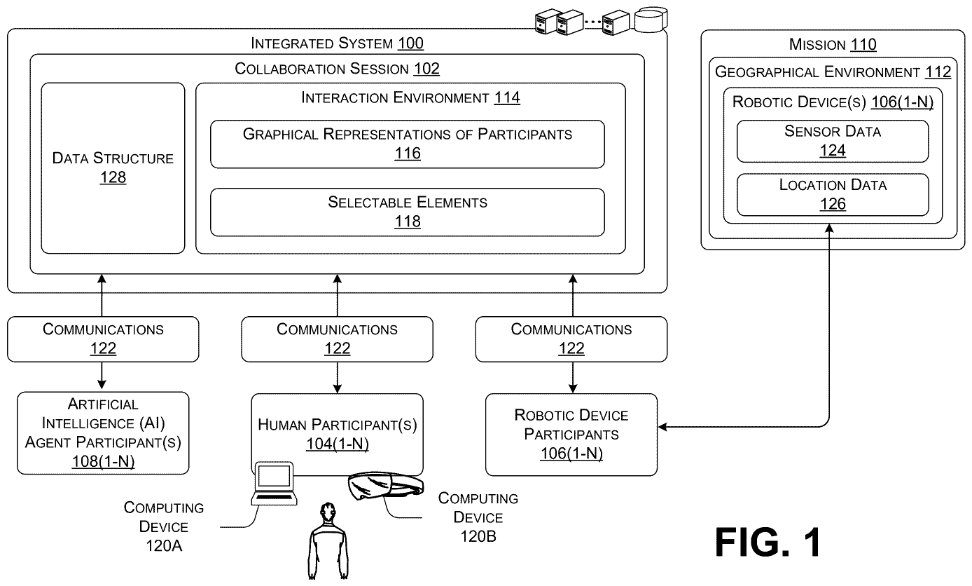

FIG. 1 illustrates an example environment in which a system 100 creates and/or executes a collaboration session 102 between one or more human participant(s) 104(1-N), one or more robotic device participant(s) 106(1-N), and one or more artificial intelligence (AI) agent participant(s) 108(1-N). The number N represents a positive integer number and can be the same or different for the human participants 104(1-N), the robotic device participants 106(1-N), and the AI agent participants 108(1-N). For example, the numbers for the different types of participants can be smaller (e.g., one, two, three, four) if the collaboration session 102 is small in scale. Alternatively, the numbers for the different types of participants can be larger (e.g., five, ten, fifteen, fifty) if the collaboration session 102 is large in scale.

The system 100 is “integrated” in the sense that it seamlessly provides common collaboration session features so that the human participant(s) 104(1-N), the robotic device participant(s) 106(1-N), and the AI agent participant(s) 108(1-N) can all work together toward a mission 110 to be completed within a geographical environment 112. That is, the collaboration session 102 may be executed in relation to the mission 110 and the geographical environment 112. The mission 110 may be related to a response to an event and a set of tasks to be completed in accordance with the mission 110 is distributed across different human and/or robotic device roles with varied responsibilities. Consequently, the robot device participants 106(1-N) reflect robotic devices that are operating and physically located in the geographical environment 112. A robotic device participant 106 is a programmable device configured to implement a series of physical actions automatically. In this context, “automatically” means the physical actions are implemented via the embedded programming of the robotic device participant 106 and/or via remote control of the robotic device participant 106 (e.g., when a human and robotic device are not co-located).

As an illustrative example, an event may be a natural disaster such as a fire from a lightning strike, a hurricane, or an earthquake. The use of robotic devices 106(1-N) can be helpful in responding to a natural disaster event. Typically, multiple organizations respond to execute and/or complete a mission 110 with the goal of helping people that are affected by the natural disaster event. For instance, first tasks for the mission 110 may be related to finding and rescuing survivors (e.g., removing people from dangerous areas). Second tasks for the mission 110 may be related to limiting further damage caused by the natural disaster event (e.g., finding areas that are burning and extinguishing the fire, securing unstable buildings). Third tasks for the mission 110 may be related to identifying damaged/offline “utility” infrastructure (e.g., electric grid infrastructure, water supply infrastructure, gas pipeline infrastructure) and fixing the damage/offline utility infrastructure so it comes back online in due time.

In this illustrative example, the multiple organizations often include different government agencies from local, state, and/or federal jurisdictions. Moreover, the multiple organizations may include private and/or charitable organizations as well. Each of the organizations may include their own personnel (e.g., the human participants 104(1-N)), their own experiences and/or procedures with respect to deploying robotic devices 106(1-N) to the geographical environment 112 in which the event occurs, as well as their own execution and/or communications infrastructure to operate the robotic devices 106(1-N). When different personnel and different types of robotic devices 106(1-N) from different organizations converge on the geographical environment 112 in response to an event such as a natural disaster, it is difficult to coordinate the tasks so that the mission can be achieved in a more effective and efficient manner.

The collaboration session 102 creates an effective and efficient solution for human participants 104(1-N) and robotic device participants 106(1-N) to coordinate the performance of the mission 110 in the geographical environment 112. Additionally, the collaboration session 102 allows for the AI agent participants 108(1-N) to assist in the coordination of the performance of the mission 110 in the geographical environment 112. For instance, via the execution of the collaboration session 102, humans from different organizations that use heterogenous robotic devices (e.g., different types of robotic devices, different types of communications) can quickly connect through a central, integrated system 100 to collaborate and coordinate performance of tasks that are intended to complete the mission 110.

The illustrative example of a natural disaster event provided above is a larger-scale event. However, it is understood in the context of this disclosure that a mission 110 can be implemented at different scales. For instance, a mission 110 can also be implemented in response to a smaller-scale event that only requires coordination and collaboration between one human participant 104, one robotic device participant 106, and one AI agent participant 108. For example, a human participant 104 may create a collaboration session 102 to coordinate with a robotic device participant 106 and/or an AI agent participant 108 to find a particular team member (e.g., determine a current location of the particular team member) in an office building so the team member can resolve a project issue a team has encountered. In this example, the event is the project issue and the mission 110 is finding the particular team member in the office building, which represents the geographical environment 112.

Consequently, the coordination enabled via a collaboration session 102 described herein may be scaled to apply in any context in which work (e.g., a mission 110) needs to be done by a human, a robotic device, and an AI agent. Various contexts include disaster response, safety and security, healthcare and medical instrumentation, manufacturing and industrial lines/warehouses, office and/or personal management, agriculture, and so forth. Accordingly, a geographical environment 112, as described herein, can include an identifiable “real-world” setting and/or area. The identifiable real-world setting and/or area can be indoor, such as an office building, a retail building, a personal residence, a warehouse, a hospital, a medical office, a factory floor, a manufacturing line, or other types of settings and/or areas within physical structures that can be blueprint-or human-defined. Alternatively, the identifiable real-world setting and/or area can be outdoor, such as a forest, a mountain, a construction site, a neighborhood, a town, a city, a county, a state, a country, a field, a pasture, or other type of outdoor settings and/or areas that can be map-or human-defined.

Robotic device participants 106(1-N) can operate on land, on water, in the air, in space, or a combination thereof, and can be programmed to perform different tasks. For example, an unmanned aerial vehicle (UAV) may be tasked with capturing video and/or dropping items from the sky. A sea drone may be tasked with capturing video and/or providing supplies to an area that cannot be reached by land. A bomb disposal robotic device may be tasked with capturing video and/or safely disabling an explosive device. A backhoe robotic device may be tasked with capturing video and/or moving dirt, rocks, and/or rubble. A dump truck robotic device may be tasked with capturing video and hauling away dirt, rocks, and/or rubble. An office or retail robotic device may be tasked with stocking retail and/or supply shelves. A warehouse robotic device may be tasked with sorting items in bins. A manufacturing robotic device may be tasked with connecting two parts of an apparatus. These example robotic devices are just a few of the numerous different types of robotic devices that have been manufactured and configured to perform various tasks in varying contexts.

A participant (e.g., a human participant 104, a robotic device participant 106, an AI agent participant 108) starts the collaboration session 102 by creating the collaboration session 102 and joining the collaboration session 102. In one example, the collaboration session 102 reflects a virtual meeting (e.g., videoconference) setting. The participant that starts the collaboration session 102 can then add other participants to the collaboration session 102 via an invitation to join. After the collaboration session 102 is started, the integrated system 100 generates an interaction environment 114 for the collaboration session 102. As shown in the examples described below, the interaction environment 114 includes a graphical representation 116 for each of the participants 104(1-N), 106(1-N), 108(1-N) that have joined the collaboration session 102. In this way, the human participants 104(1-N) can view and/or interact with various resources (e.g., robotic device participants 106(1-N), AI agent participants 108(1-N)) that are available and/or deployed to assist in completion of the mission 110.

Additionally, the interaction environment 114 includes selectable element(s) 118 that enable a human participant 104 to switch between at least two viewing states associated with the participants in the collaboration session 102, examples of which are described herein. The system 100 then provides the interaction environment 114 to a computing device 120A-B associated with a human participant 104. In one example, the interaction environment 114 is displayed on a computing screen in two-dimensions, and thus, the computing device 120A can be a desktop computer, a gaming device, a tablet computer, a personal data assistant (PDA), a laptop computer, a telecommunication device (e.g., a smartphone), a wearable device (e.g., a smartwatch), an automotive computer, a network-enabled television, or any other sort of computing device capable of displaying the interaction environment in two dimensions. In another example, the interaction environment 114 is displayed in an immersive environment that includes more than two dimensions (e.g., a 3D environment), and thus, the computing device 120B can be a virtual reality (VR) computing device, an augmented reality (AR) computing device, or a mixed reality (MR) computing device.

FIG. 1 further illustrates that the system 100 enables communications 122 between the collaboration session 102 and each of the participants 104(1-N), 106(1-N), 108(1-N) that have joined the collaboration session 102. In one example, the communications 122 allow for the robotic device participants 106(1-N) to transmit sensor data 124 and/or location data 126 to the collaboration session. The sensor data 124 can include one or more of image data (e.g., still images) captured by an image capture device embedded in or attached to the robotic device participant, video data (e.g., a sequence of video frames) captured by a video capture device embedded in or attached to the robotic device participant, audio data captured by a microphone embedded in or attached to the robotic device participant, temperature data captured by a thermometer embedded in or attached to the robotic device participant, air quality data captured by an air quality sensor embedded in or attached to the robotic device participant, pressure data captured by a pressure sensor embedded in or attached to the robotic device participant, velocity data captured by a velocity sensor embedded in or attached to the robotic device participant, smoke data captured by a smoke detecting sensor embedded in or attached to the robotic device participant, gas data captured by a gas detecting sensor embedded in or attached to the robotic device participant, thermal data captured by a thermal sensor embedded in or attached to the robotic device participant, depth data captured by a depth sensor embedded in or attached to the robotic device participant, odor (smell) data captured by an odor sensor embedded in or attached to the robotic device participant, lidar data captured by a laser component embedded in or attached to the robotic device participant, radar data captured by a radar component embedded in or attached to the robotic device participant, or infrared (IR) data captured by an IR sensor embedded in or attached to the robotic device participant. While a list of example types of data and/or sensors is provided above, it is understood in the context of this disclosure, that a robotic device participant 106 can be configured with hardware, firmware, and/or software to detect and/or sense any type of environmental data. The location data 126 can reflect a location, or position, of a robotic device participant 106 in the geographical environment 112 (e.g., a Global Positioning System (GPS) location).

In another example, the communications 122 allow for the human participants 104(1-N) to transmit and/or receive individual streams of data corresponding to the participants 104(1-N), 106(1-N), 108(1-N), such as audio and/or visual data that capture the appearance and speech of a participant in the collaboration session, a video stream, or video feed, from a camera embedded on a robotic device, and so forth.

In yet another example, the communications 122 allow for the AI agent participants 108(1-N) to receive a context of the interaction environment 114 in a consumable format (e.g., code-based format), as stored in a data structure 128 for the collaboration session 102. Access to the context of the whole interaction environment 114, or a particular aspect of the interaction environment 114 (e.g., a video stream from a robotic device participant 106) enables an AI agent participant 108 to understand and/or analyze particular characteristics of the collaboration session 102.

Consequently, regardless of the size and/or scope of the mission 110 and/or a scale of an event to which the mission 110 responds, the collaboration session 102 described herein enables the different types of participants 104(1-N), 106(1-N), 108(1-N) to work together to complete the mission 110. The collaboration session 102 presents a low barrier of entry for humans and/or robotic devices to be part of a coordinated mission 110. Moreover, the collaboration session 102 enables the integration of heterogenous robotic devices (e.g., different fleets of robotic devices) that are not designed and/or configured to communicate with one another. Moreover, through the use of the accessible AI agents, the collaboration session 102 enables effective participation for humans without detailed working knowledge of the robotic devices deployed to the geographical environment 112 in which the mission 110 is being implemented, thereby reducing the cognitive load required for successful missions and increasing the overall efficiency for mission completion.

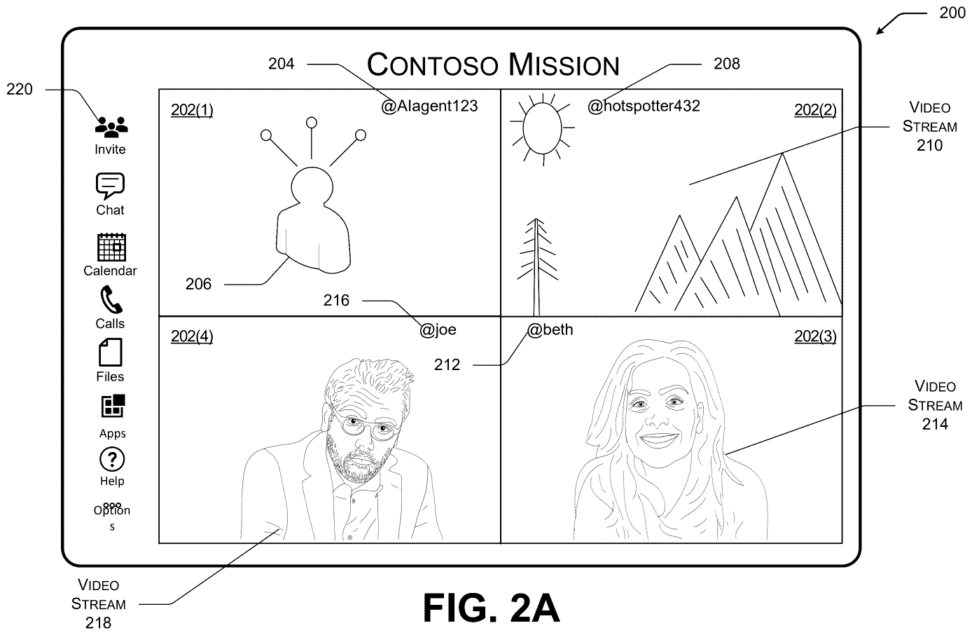

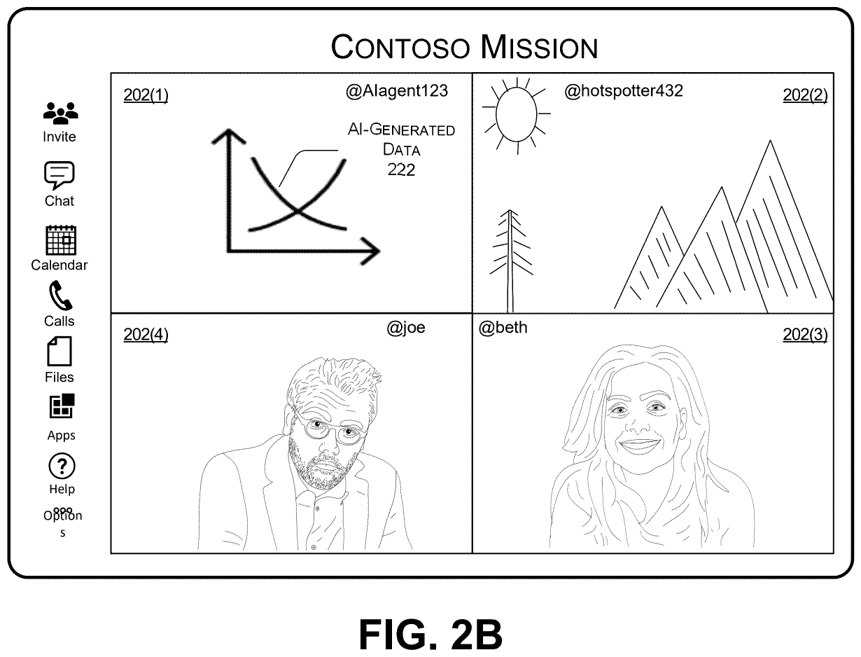

FIG. 2A illustrates an example interaction environment 200 (e.g., interaction environment 114) where graphical representations for at least one human participant, at least one robotic device participant, and at least one artificial intelligence agent participant are displayed (e.g., via computing device 120A-B). As shown, the mission 110 is entitled the “Contoso Mission”, which is directed to the example context of responding to a forest fire event. Accordingly, the interaction environment 200 includes display areas 202(1-4) that display graphical representations 116 for four participants. Additionally, the display areas 202(1-4) display identifiers for the four participants. For example, display area 202(1) shows that an AI agent participant 108 identified as “@AIagent123” 204 has joined the collaboration session 102 and the display area 202(1) includes a graphical representation 206 of “@AIagent123” 204. Display area 202(2) shows that a robotic device participant 106 identified as “@hotspotter 432” 208 has joined the collaboration session 102 and the display area 202(2) includes a graphical representation 210 of “@hotspotter 432” 208 in the form of a video stream being captured by a video capture component embedded in or attached to “@hotspotter432”208. Display area 202(3) shows that a human participant 104 identified as “@beth” 212 has joined the collaboration session 102 and the display area 202(3) includes a graphical representation 214 of “@beth” 212 in the form of a video stream being captured by a video camera on Beth's computing device 120A. Display area 202(4) shows that a human participant 104 identified as “@joe” 216 has joined the collaboration session 102 and the display area 202(4) includes a graphical representation 218 of “@joe” 216 in the form of a video stream being captured by a video camera on Joe's computing device 120A.

FIG. 2A further illustrates a selectable element 220 that enables a human participant 104 (e.g., Joe or Beth) to invite not only other human participants, but AI agents and robotic devices as well. Consequently, via the interaction environment 200 provided by a collaboration session 102, human participants 104(1-N) are provided with a centralized space that allows the human participants to not only view helpful resources (e.g., robotic device participants 106(1-N), AI agent participants 108(1-N)) that are available and/or deployed to assist in completion of the mission 110, but also interact with the helpful resources in a way that provides effective and efficient coordination.

FIG. 2B illustrates the interaction environment 200 of FIG. 2A with a viewing state that has been switched. In this example, the AI agent participant 108 identified as “@AIagent123” 204 has been called upon to process (e.g., interpret, understand) the video stream 210 provided by the robotic device participant 106 identified as “@hotspotter432” 208. Accordingly, the viewing state of the interaction environment 200 has switched with respect to the graphical representation of the AI agent participant 108 identified as “@AIagent123” 204. That is, a personification of the AI agent participant 108 identified as “@AIagent123” 204 (as shown in FIG. 2A) is replaced with meaningful AI-generated data 222. The AI agent participant 108 identified as “@AIagent123” 204 functions as a translation and/or orchestration interface between humans and robotic devices.

FIGS. 2A-B include a smaller number of graphical representations for a smaller number of respective participants in a collaboration session 102. However, it is understood in the context of this disclosure that a collaboration session 102 can have a larger number of participants (e.g., twenty participants, thirty participants, fifty participants) depending on the size and/or scope of the mission 110.

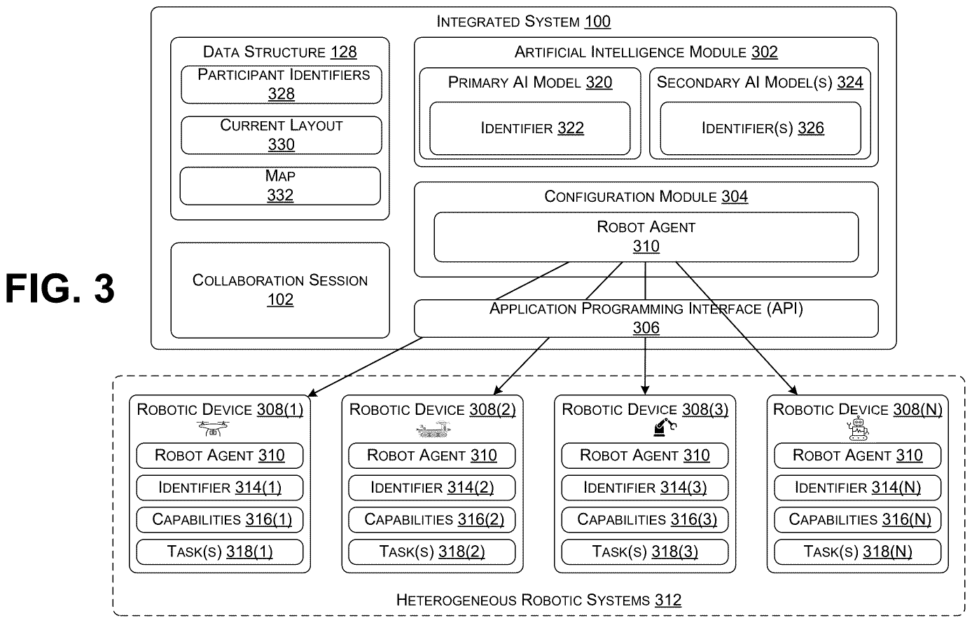

FIG. 3 illustrates further aspects of the integrated system 100 executing a collaboration session 102. The integrated system 100 includes an artificial intelligence (AI) module 302 and a configuration module 304. The functionality described herein in association with the illustrated modules can be performed by a fewer number of modules or a larger number of modules on one device (e.g., server) in the integrated system 100 or spread across multiple devices in the integrated system 100.

The configuration module 304 is configured to expose an application programming interface (API) 306 that allows different types of robotic devices 308(1-N) to access and download a robot agent 310 that enables robot device participation in the collaboration session 102. The robot agent 310 includes centralized code (e.g., a software development kit, application programming interface(s)) that configures the different types of robotic devices 308(1-N) with communication software that is compatible with the collaboration session 102. That is, after downloading and installing the robot agent 310, a robotic device 308 can join and participate in the collaboration session 102 via the communication (e.g., transmission) of robot data (e.g., sensor data 124 and/or location data 126).

Thus, the robot agent 310 made available by the configuration module 304 via the API 306 configures a bi-directional communication bridge between a robotic device 308 and the collaboration session 102. More specifically, this bi-directional communication bridge connects the robotic device 308 to cloud infrastructure that hosts the collaboration session 102 via different types of networks including private and/or public local area networks (LANs), private and/or public metropolitan area networks (MANs), private and/or public wide area networks (WANs), Wi-Fi networks, public and/or private mobile networks (e.g., 5G networks, LTE networks), satellite networks, radio networks, and so forth.

As illustrated in FIG. 3, the robotic devices 308(1-N) are heterogeneous 312 robotic devices. More specifically, the robotic devices 308(1-N) respectively include identifiers 314(1-N) that can either define, or be mapped to, different hardware, firmware, and/or software components. As shown, robotic device 308(1) has an identifier 314(1) associated with a first set of capabilities 316(1) with respect to hardware, firmware, and/or software components of an unmanned aerial vehicle (UAV) configured to perform particular task(s) 318(1). Robotic device 308(2) has an identifier 314(2) associated with a second set of capabilities 316(2) with respect to hardware, firmware, and/or software components of a track crawler robotic device configured to perform particular task(s) 318(2). Robotic device 308(3) has an identifier 314(3) associated with a third set of capabilities 316(3) with respect to hardware, firmware, and/or software components of an arm-based robotic device configured to perform particular task(s) 318(3) (e.g., pick up and move an object). Robotic device 308(N) has an identifier 314(N) associated with a Nth set of capabilities 316(N) with respect to hardware, firmware, and/or software components of a humanoid robotic device configured to perform particular task(s) 318(N). A version of the robot agent 310 downloaded and installed on the robotic devices 308(1-N) can be a common version. Alternatively, a version of the robot agent 310 downloaded and installed on the robotic devices 308(1-N) can be a customized version (e.g., the centralized code has been tailored based on an identifier 314).

In various examples, the invitation to join the collaboration session 102 is a notification that wakes a robotic device 308 from a sleep state and/or activates the central code 310 to enable the bi-directional communication bridge to/from the collaboration session 102. As described above, after a robotic device 308 has joined the collaboration session, the robotic device can start participating by communicating (e.g., reporting) sensor data 124 and/or location data 126 to the collaboration session 102.

The AI module 302 provides the collaboration session 102 access to an intelligence backbone in the form of AI models (e.g., multi-modal generative-AI models, large language models (LLMs), small language models (SLMs)). In various examples, the AI module 302 includes a primary AI model 320 and associated identifier 322. The primary AI model 320 can perform general intelligence support for the interaction environment 114. Furthermore, the primary AI model 320 can serve as a conduit between humans and secondary AI model(s) 324 with associated identifier(s) 326. The secondary AI model(s) 324 can be tailored to perform more specific processing and/or analysis. For example, each type of robotic device 308(1-N) may have a dedicated secondary AI model 324 to assist with task(s) 318(1-N). Thus, after the robotic devices 308(1-N) join the communication session 102, the primary AI model 320 can recommend that corresponding secondary AI models 324 dedicated to the robotic devices 308(1-N) be added or invited to the collaboration session 102. In various examples, AI processing can occur anywhere within a distributed, cloud environment. That is, the AI process can occur at a robotic device 308 (e.g., via a small language model implemented in the robot agent 110), at an edge location, or in the cloud.

In some instances, the primary AI model 320 and/or the secondary AI model(s) 324 comprise large action models (LAMs) and/or small action models (SAMs) that work in combination with other pre-trained or customized models, such as LLMs, SLMs, large multimodal models, and/or small multimodal models. While language models have the main function of generating text, action models can generate and/or perform concrete actions with a given set of instructions or commands from a human participant. Consequently, the AI agent participants 108(1-N) can use action models to act like humans in terms of analyzing data and then acting based on the analysis. For example, while a language model (e.g., LLM, SLM) might be used to understand and respond to a chat message, an action model (e.g., a LAM, a SAM) could autonomously generate and perform tasks described by the chat message. Consequently, action models are sophisticated components that help an AI agent participant 108 understand and execute complex tasks.

In various examples, components of an action model include a foundational language model, as well as a reinforcement learning from human feedback (RLHF) component or a direct preference optimization (DPO) component to fine tune the foundational language model (e.g., make the foundational language model more accurately understand different areas or topics). The language model is then connected to an external tool (e.g., a robotic device participant) that perform actions on its own, which essentially turns the language model into an action model. Consequently, action models are configured to interact with various systems and/or interfaces to perform tasks that involve actual actions, such as controlling robotic device participants.

Further shown in FIG. 3 is the data structure 128 for the collaboration session 102 and/or interaction environment 114. Again, the data structure 128 includes code reflecting the context of the collaboration session 102 and/or interaction environment 114. To this end, the data structure 128 includes participant identifiers 328, a current layout 330 of the interaction environment 114, and a map 332 of the geographical environment 112, each of which is further discussed herein.

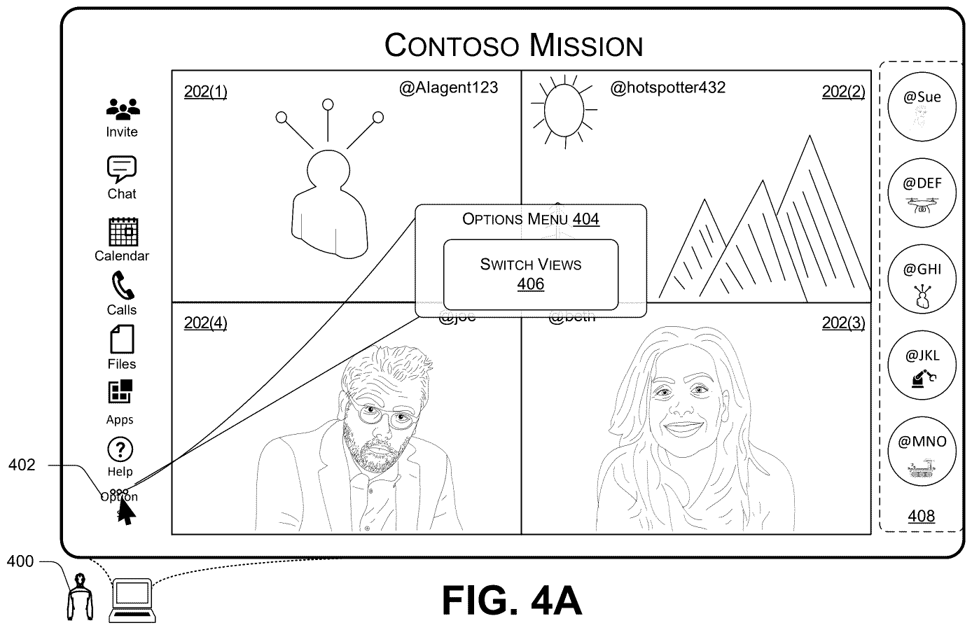

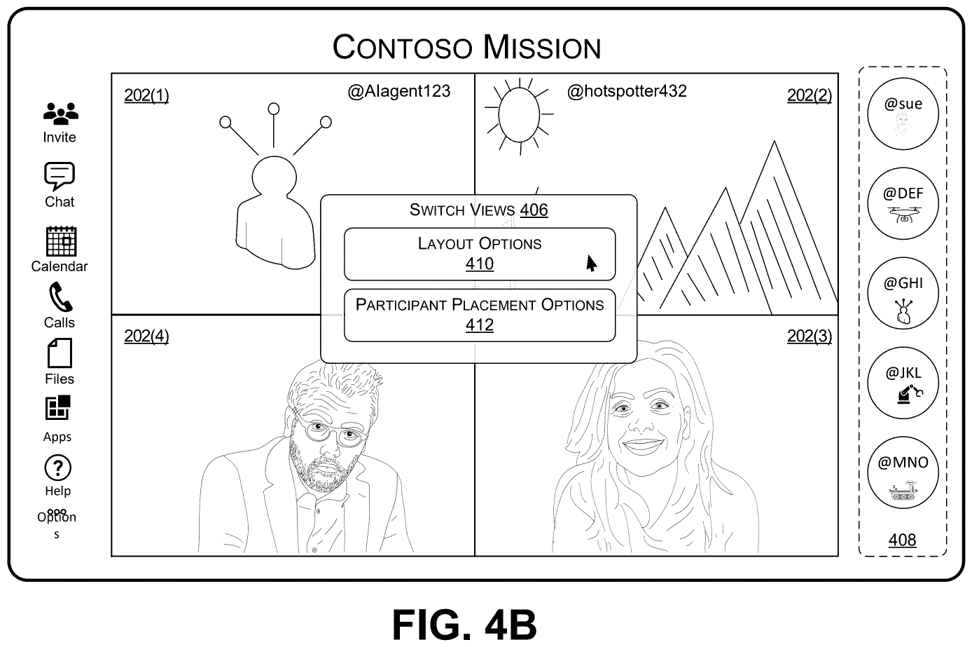

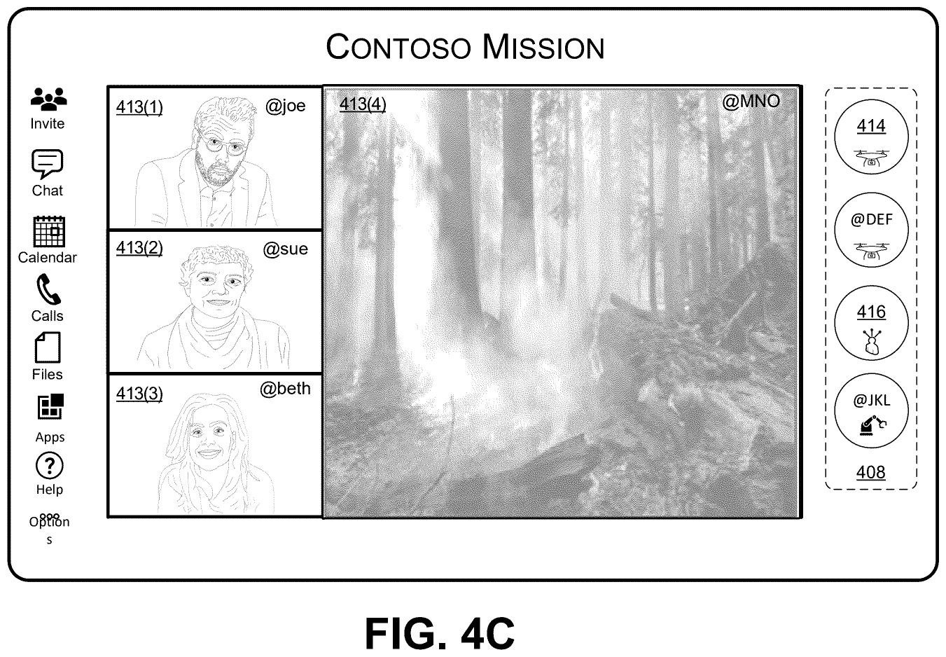

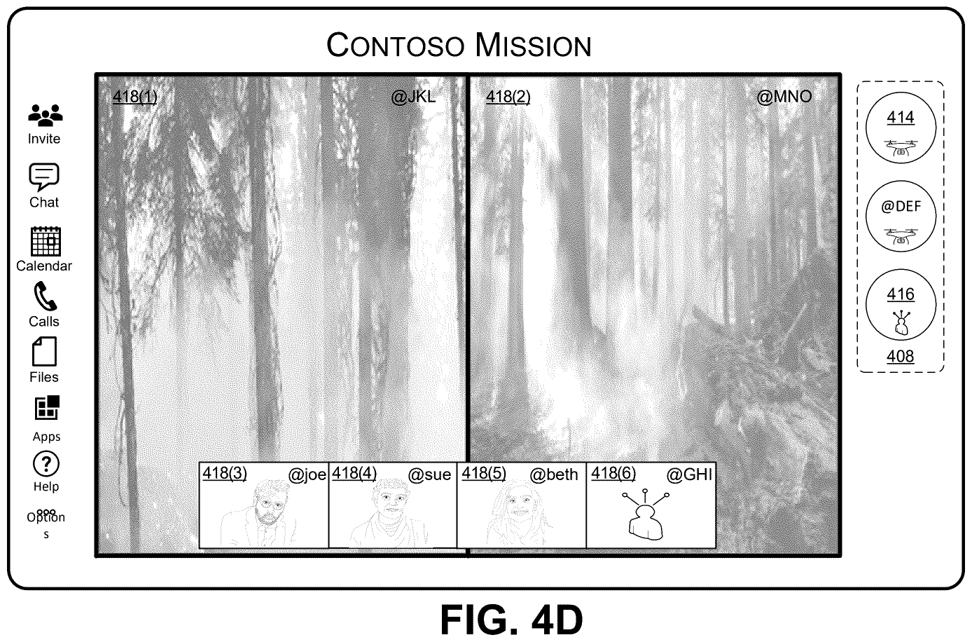

FIG. 4A illustrates the example interaction environment 200 of FIG. 2A, where a human participant 400 in the collaboration session 102 that is viewing the interaction environment 200 is using a cursor to provide input that selects an element 402 providing access to an options menu 404 associated with a collaboration session 102. As shown, the options menu 404 at least includes a selectable element 406 to switch view states of the interaction environment 200. In one example, switching view states changes the participants that are displayed in primary areas. That is, in the interaction environment 200, the areas 202(1-4) are designated as “primary” areas and the participants graphically represented and identified therein are referred to as “primary” participants. As participation in the collaboration session 102 grows, the number of participants increases and due to limitations on a number of primary areas in the interaction environment 200, a secondary area 408 can be added to the interaction environment 200 to include graphical representations 116 of secondary participants. A secondary participant is one that has joined the collaboration session 102 but is not the focus of the collaboration session 102 at a current time.

As shown, the secondary area 408 includes graphical representations for “@sue”, “@DEF”, “@GHI”, “@JKL”, and “@MNO”. These graphical representations 116 in the secondary area 408 indicate a type of participant (e.g., a human participant, a robotic device participant, an AI agent participant) and/or a type of robotic device via an icon contained therein. Consequently, the human participant 400 viewing the interaction environment 200 in FIG. 4A is readily aware of the resources that have been available to assist with completion of a mission 110. The secondary area 408 is typically on the peripheral (e.g., edge) of the interaction environment 200. While the secondary area 408 is on the right side of the interaction environment 200 in FIG. 4A, it is understood in the context of this disclosure that the secondary area 408 can alternatively be on the top of the interaction environment 200, on the bottom of the interaction environment 200, or on the left side of the interaction environment 200.

Continuing on, FIG. 4B illustrates the interaction environment where the human participant 400 is providing input to switch a view state with respect to a layout and primary participants. Upon selection of the selectable element 406 to switch view states in FIG. 4A, the human participant 400 is presented with selectable elements representing layout options 410 and/or selectable elements representing participant placement options 412.

FIG. 4C illustrates a switch to a new view state based on human input selecting elements 410 and/or 412 in FIG. 4B. As shown, the layout of the areas 413(1-4) designated as primary has changed and further the human participant “@sue” and the robotic device participant “@MNO” have been elevated to primary participants. In response to the human participant “@sue” and the robotic device participant “@MNO” being elevated (e.g., via human selection such as a drag and drop input) to primary participants in primary areas 413(1-4), the robotic device participant “@hotspotter432” (a “UAV” represented by element 414 in FIG. 4C) and the AI agent participant “@AIagent123” (represented by element 416 in FIG. 4C) have been moved to the secondary area 408. So here, the human participant 400 provides input via elements 410 and/or 412 to view the video stream from a different robotic device participant “@MNO”, compared to the video stream from robotic device participant “@hotspotter432” shown in FIG. 4B. The human participant “@sue” may have been elevated to a primary area 413(3) because she is the person responsible for the robotic device participant “@MNO”.

FIG. 4D illustrates an example switch to another new view state based on human input selecting elements 410 and/or 412 in FIG. 4B. As shown, the layout of the areas 418(1-6) designated as primary has changed, as video streams from the robotic device participants “@JKL” and “@MNO” are prominently displayed in primary areas 418(1-2), and further the human participant “@sue” and the AI agent participant “@GHI” have also been elevated to primary participants in primary areas 418(4) and 418(6), respectively. In response, the robotic device participant “@hotspotter432” (represented by element 414) and the AI agent participant “@AIagent123” (represented by element 416) have been moved to the secondary area 408. So here, the human participant 400 provides input via elements 410 and/or 412 to view the video streams from both robotic device participants “@JKL” and “@MNO” at the same time.

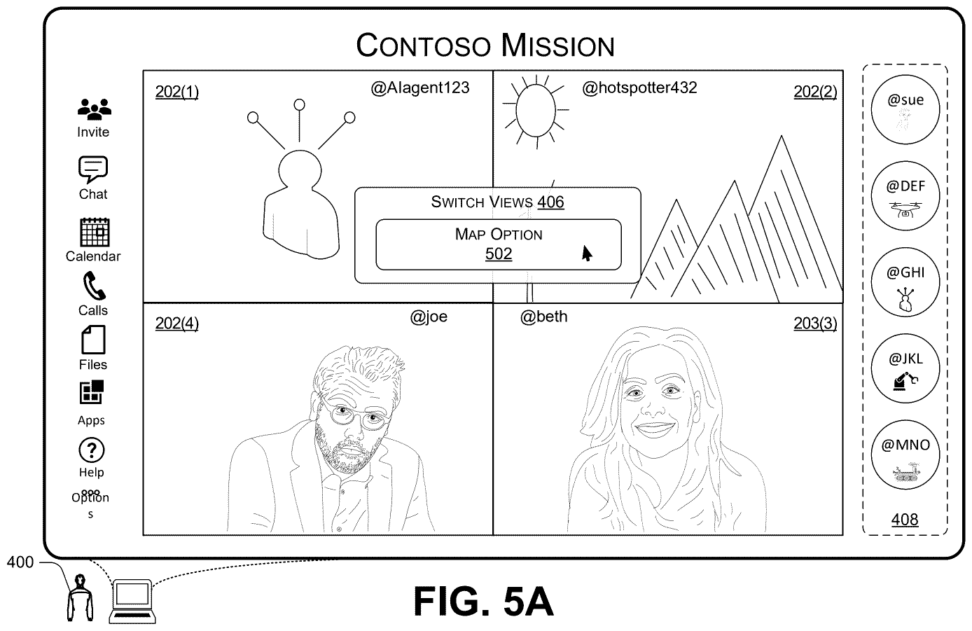

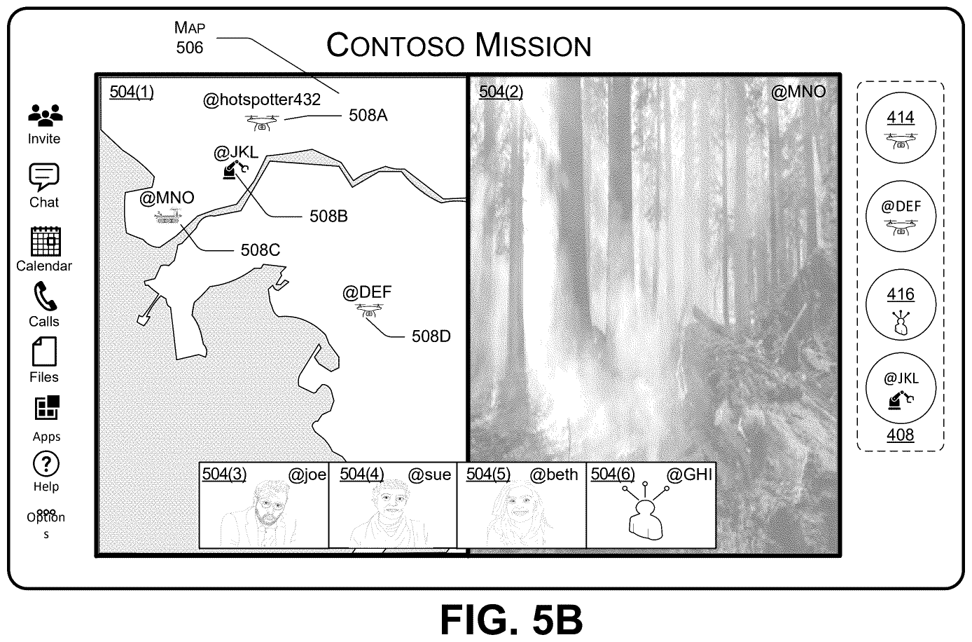

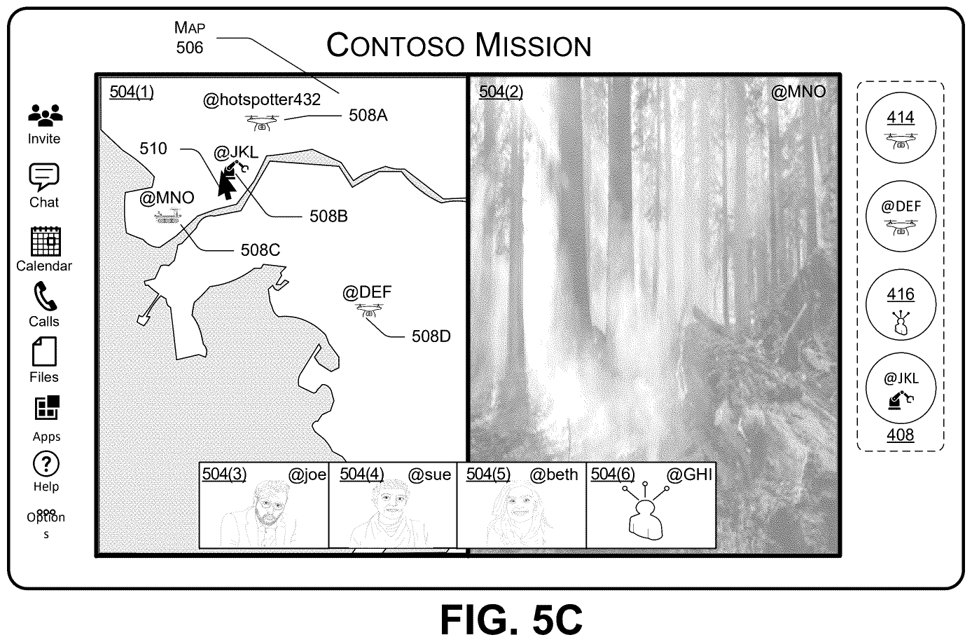

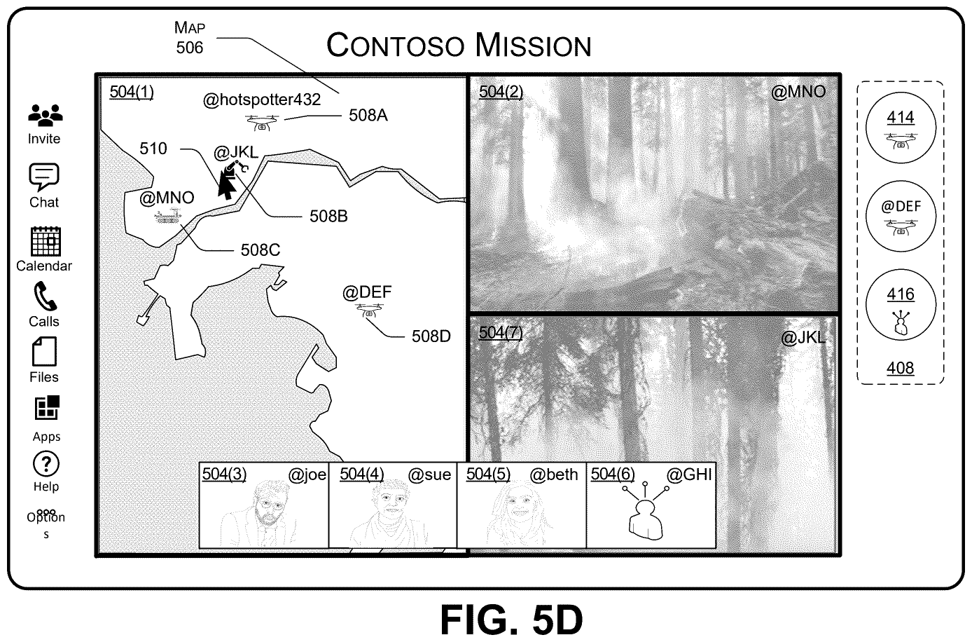

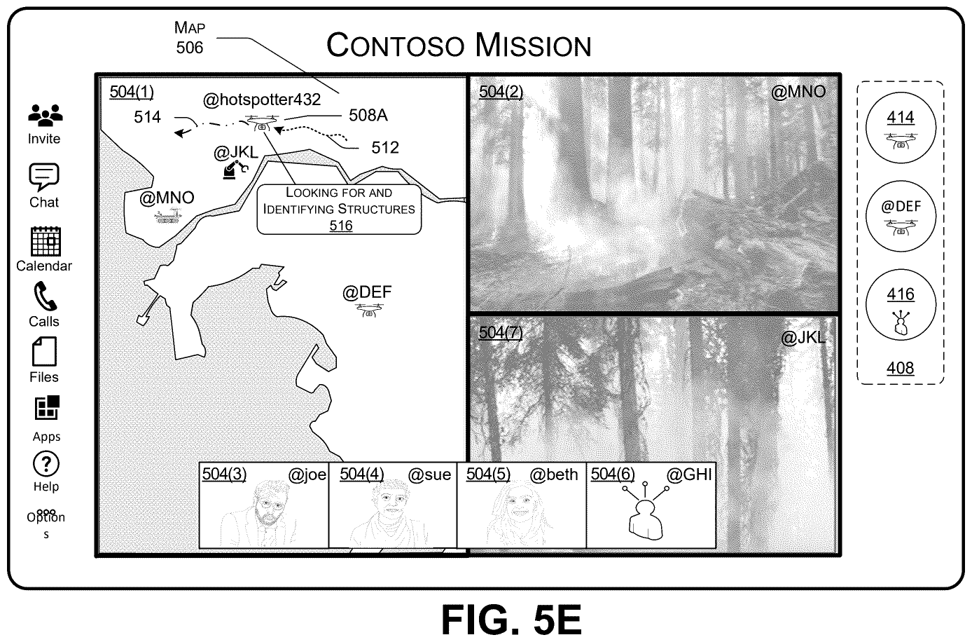

FIG. 5A returns to the example interaction environment 200 of FIG. 2A and as further discussed in FIG. 4A, where the human participant 400 provides input to switch a view state. In this example, switching view states introduces a map of the geographical environment 200. Accordingly, upon selection of the selectable element 406 to switch view states in FIG. 4A, the human participant 400 is presented with a selectable element 502 representing a map option.

FIG. 5B illustrates a switch to a new view state based on the input selecting element 502 in FIG. 5A. As shown, the layout of the interaction environment 200 has changed and now includes areas 504(1-6) designated as primary. Moreover, a map 506 of the geographical environment 112 is now presented in primary area 504(1) of the interaction environment 200 alongside a video stream from robotic device participant “@MNO” in primary area 504(2). Further, the human participant “@sue” and the AI agent participant “@GHI” have also been elevated to primary participants in primary areas 504(4) and 504(6), respectively.

The map 506 includes icons and/or identifiers 508A-D that represent the real-world, physical locations of the robotic device participants (e.g., “@hotspotter432”, “@JKL”, “@MNO”, “@DEF”) that have been deployed to operate in the geographical environment 112. Consequently, human and/or AI agent participants can gain an understanding of the geographical environment 112 to better coordinate a response using the robotic device participants that have been deployed.

FIG. 5C illustrates how the human participant 400 can move a cursor 510 to an icon associated with a robotic device participant on the map 506, and select the icon. Thus, the icons 508A-D are selectable elements, and as shown, the human participant 400 provides input to select the icon 508B associated with the robotic device participant “@JKL” in the map 506. Moving to FIG. 5D, a different view state based on the input in FIG. 5C is shown. More specifically, based on the selection of the icon 508B representing the robotic device participant “@JKL” in FIG. 5C, the video stream from robotic device participant “@JKL” is now shown in a newly added primary area 504(7) below the video stream from the robotic device participant “@MNO”. In an alternative example, the video stream from robotic device participant “@JKL” can replace the video stream from the robotic device participant “@MNO” in the primary area 504(2) of FIGS. 5B-5C based on the selection of the icon 508B representing the robotic device participant “@JKL”.

FIG. 5E illustrates how the map 506 can further show other data related to the robotic device participants. As an example, the map 506 can include a graphical element 512 that represents previous movement in the geographical environment 112 for the robotic device participant “@hotspotter432” represented by icon 508A. The map 506 can include a graphical element 514 that represents an orientation and/or future movement (as programmed) for the robotic device participant “@hotspotter432” represented by icon 508A. Additionally, the map 506 can include a graphical element that shows a task currently being performed by a robotic device participant. In the example of FIG. 5E, the map 506 shows a graphical element 516 indicating that “@hotspotter432” represented by icon 508A is currently looking for and/or identifying physical structures that are in danger due to a fast moving forest fire.

In various examples, the example switches being viewing states described above with respect to FIGS. 4A-D and 5A-E can only be implemented by humans with defined control privileges (e.g., a host or creator of the collaboration session, a designated lead from each organization participating). As a number of participants in a collaboration session 102 scales (e.g., increases), the ability for a small number of human participants to oversee and control the viewing state can become paramount. Further, all the human participants 104(1-N) in the collaboration session 102 see the same content via a common view state of the interaction environment, in various embodiments. However, in other embodiments, the human participants 104(1-N) in the collaboration session 102 have the ability to decouple from the common view state and customize the viewing state to their own preference.

The human inputs described above with respect to FIGS. 4A-D and 5A-E are based on selection of graphical elements (e.g., via touch and/or mouse movement of a user-controlled element such as a cursor). However, it is understood in the context of this disclosure that the human inputs that interact with graphical elements and/or perform functions can be provided via alternative means. For example, a human input can be provided to activate a graphical element and/or perform a function via a key press or sequence of key presses via a keyboard. A human input can be provided to activate a graphical element and/or perform a function via a voice command. A human input can be provided to activate a graphical element and/or perform a function via a gesture that is recognized in a three-dimensional space.

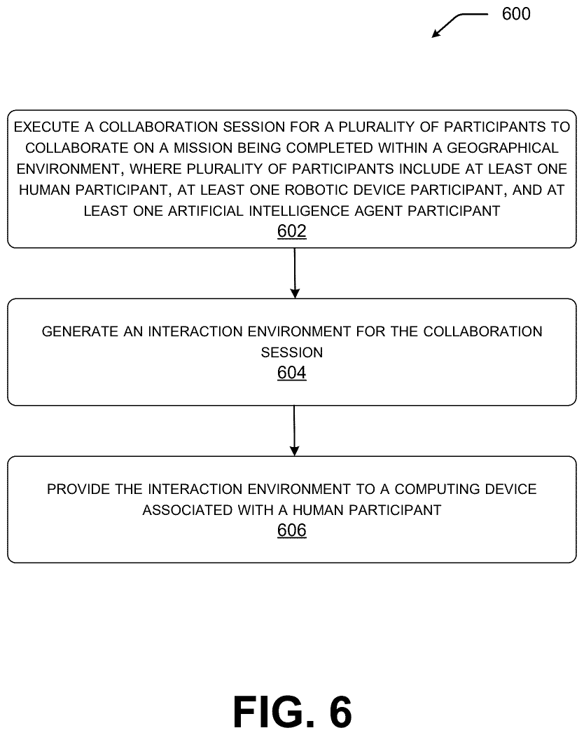

Proceeding to FIG. 6, a process 600 for executing a collaboration session between at least one human participant, at least one robotic device participant, and at least one artificial intelligence agent participant is shown and described. The process 600 begins at operation 602 where a system executes a collaboration session for a plurality of participants to collaborate on a mission being completed within a geographical environment. As described above, the plurality of participants includes a human participant, a robot device participant, and an artificial intelligence agent participant. The robotic device participant is located in the geographical environment and the collaboration session is configured to receive data from the robotic device participant.

At operation 604, the system generates an interaction environment for the collaboration session. As described above, the interaction environment includes a graphical representation for each of the plurality of participants and an interactive (e.g., selectable) element that enables the human participant to switch between at least two viewing states associated with the plurality of participants.

At operation 606, the system provides the interaction environment to a computing device associated with the human participant. Consequently, the human participant can gain an understanding of the geographical environment to better coordinate a response using available resources (e.g., AI agent participants, robotic device participants that have been deployed to the geographical environment).

For ease of understanding, the processes discussed in this disclosure are delineated as separate operations represented as independent blocks. However, these separately delineated operations should not be construed as necessarily order dependent in their performance. The order in which the processes are described is not intended to be construed as a limitation, and any number of the described process blocks may be combined in any order to implement the processes or an alternate processes. Moreover, it is also possible that one or more of the provided operations is modified or omitted.

The particular implementation of the technologies disclosed herein is a matter of choice dependent on the performance and other requirements of a computing device. Accordingly, the logical operations described herein are referred to variously as states, operations, structural devices, acts, or modules. These states, operations, structural devices, acts, and modules can be implemented in hardware, software, firmware, in special-purpose digital logic, and any combination thereof. It should be appreciated that more or fewer operations can be performed than shown in the figures and described herein. These operations can also be performed in a different order than those described herein.

It also should be understood that the illustrated processes can end at any time and need not be performed in their entirety. Some or all operations of the processes, and/or substantially equivalent operations, can be performed by execution of computer-readable instructions included on a computer-storage media, as defined below. The term “computer-readable instructions,” and variants thereof, as used in the description and claims, is used expansively herein to include routines, applications, application modules, program modules, programs, components, data structures, algorithms, and the like. Computer-readable instructions can be implemented on various system configurations, including single-processor or multiprocessor systems, minicomputers, mainframe computers, personal computers, hand-held computing devices, microprocessor-based, programmable consumer electronics, combinations thereof, and the like.

Thus, it should be appreciated that the logical operations described herein are implemented (1) as a sequence of computer implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as states, operations, structural devices, acts, or modules. These operations, structural devices, acts, and modules may be implemented in software, in firmware, in special purpose digital logic, and any combination thereof.

For example, the operations of the processes can be implemented, at least in part, by modules running the features disclosed herein can be a dynamically linked library (DLL), a statically linked library, functionality produced by an application programing interface (API), a compiled program, an interpreted program, a script, or any other executable set of instructions. Data can be stored in a data structure in one or more memory components. Data can be retrieved from the data structure by addressing links or references to the data structure.

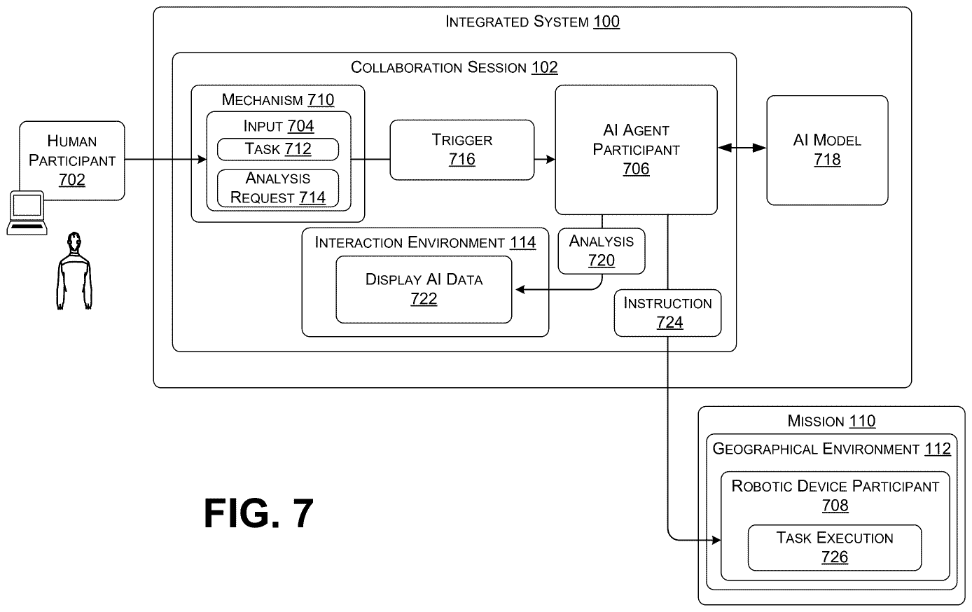

FIG. 7 illustrates further aspects of the integrated system 100 executing the collaboration session 102, as described above with respect to FIGS. 1 and 3. FIG. 7 is directed to enabling a human participant 702 (e.g., one of human participants 104(1-N)) to provide input 704 that causes an AI agent participant 706 (e.g., one of AI agent participants 108(1-N)) to perform an analysis associated with an aspect of the interaction environment 114 and/or to generate and transmit an instruction to a robotic device participant 708 (e.g., one of robotic device participants 106(1-N)) that is deployed to a geographical environment 112 to assist with completion of a mission 110.

As shown, via execution of the collaboration session 102, the integrated system 100 exposes a mechanism 710 for the human participant 702 to provide the input 704. As further described herein, the mechanism 710 is configured to receive text-based and/or voice inputs 704. In one example, the input 704 describes a task 712 that is part of the mission 110 and that causes the artificial intelligence agent participant 706 to assist with completion of the task 712 that is part of the mission 110. In another example, the input 704 comprises a request 714 for the artificial intelligence agent participant 706 to analyze information (e.g., sensor and/or location data received from the robotic device participants operating in a geographical environment 112).

Accordingly, the mechanism 710 receives the input 704 from the human participant 702 which causes the collaboration session 102 to trigger 716 (e.g., call on) the AI agent participant 706. The input 704 may be provided in the form of a prompt (e.g., entered via text or spoken via a voice command). In examples described herein, the prompt may be an instructional prompt that directs the AI agent participant 706 to perform a specific task 712 or an interpretive prompt that asks the AI agent participant 706 to interpret or analyze information. Alternatively, the prompt may be a generative prompt that requests the AI agent participant 706 to create new content such as text or images.

After being triggered 716, the AI agent participant 706 uses a corresponding AI model 718 (e.g., primary AI model 320 or a secondary AI model 324) to act in accordance with the input 704. That is, the AI agent participant 706 can perform an analysis 720 of information associated with the collaboration session 102 and/or interaction environment 114 and display AI data 722 associated with the analysis 720 via the interaction environment 114.

Alternatively, the AI agent participant 706 can generate an instruction 724 and transmit, via the collaboration session 102, the instruction 724 to the robotic device participant 708. In various examples, the instruction 724 is generated and transmitted via a file that includes text and/or executable code in a format that is understood by the robotic device participant 708 such that the robotic device participant 708 can execute 726 the task 712 described in the input 704 received from the human participant 702.

Consequently, the collaboration session 102 enables a human participant 702 to simply indicate their intent through an input 704 in order to trigger an AI agent participant 706 to perform a more complex analysis 720 and/or to generate and transmit a more complex instruction 722 to a robotic device participant 708. Stated alternatively, the collaboration session 102 enables effective participation for humans without detailed working knowledge of the robotic devices deployed to the geographical environment 112 in which the mission 110 is being implemented, thereby reducing the cognitive load required for successful missions and increasing the overall efficiency for mission completion.

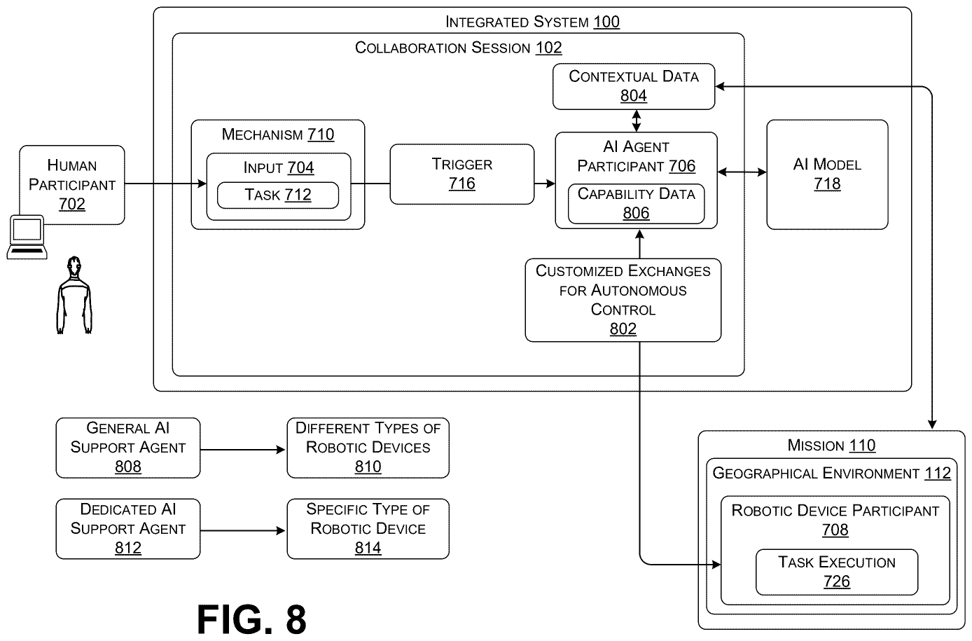

FIG. 8 illustrates further aspects of the integrated system 100 executing the collaboration session 102 in FIG. 7. In FIG. 8, the AI agent participant 706 is able to access and use two types of data to generate the instruction 724 of FIG. 7. As described in further examples below, these two types of data ultimately enable the AI agent participant 706 to autonomously (e.g., without further human input beyond the original input 704) control the robotic device participant 708 via a customized set of exchanges 802.

Specifically, the AI agent participant 706 is configured to access, via the collaboration session 102, contextual data 804 that defines aspects associated with at least one of the geographical environment 112 and/or the mission 110. As a specific example, if an input 704 states “Joe is thirsty” then the mission 110 is associated with locating and delivering a drink to Joe so Joe can quench the thirst. In this example, Joe is the human participant 702 and the AI agent participant 706 can determine, via the contextual data 804, that Joe is an employee that works in office “123” of building “XYZ” (e.g., the geographical environment 112). Moreover, the AI agent participant 706 can determine, via the contextual data 804, that Joe typically drinks coffee in the morning and soda in the afternoon. Accordingly, depending on the time when Joe initiates the collaboration session 102, the AI agent participant 706 generates an instruction 724 for the robotic device participant 708 to retrieve either a coffee (e.g., if the collaboration session 102 is initiated in the morning) or a soda (e.g., if the collaboration session 102 is initiated in the afternoon) from one of the break areas in building “XYZ” and deliver the coffee or the soda to office “123” for Joe.

Additionally, the AI agent participant 706 is configured to access, via the collaboration session 102, capability data 806 for the robotic device participant 708. As discussed above with respect to FIG. 3, the AI agent participant 706 can use an identifier associated with the robotic device participant 708 to determine its capabilities with respect to hardware, firmware, and/or software components. In one example, the AI agent participant 706 uses the identifier to obtain known information about the robot from public or other available sources (e.g., a “Spec Sheet” or an “Operating Manual”) and extracts the capability data 806 from the known information. Accordingly, the AI agent participant 706 can learn what the robotic device participant 708 is capable and/or incapable of doing. Moreover, the AI agent participant 706 can generate customized instructions to ensure the robotic device participant 708 can successfully execute the task 726 given its capabilities and/or incapabilities outlined in the capability data 806.

Continuing the specific example above, assume the capability data 806 for the robotic device participant 708 indicates that the robotic device participant 708 is incapable of opening a cooling unit such as a refrigerator via a sliding or pulling motion to pick up a can of soda. Moreover, assume that the capability data 806 for the robotic device participant 708 indicates that the robotic device participant 708 is capable of placing a cup under a soda machine and pressing a button to fill the cup. Accordingly, the AI agent participant 706 can use this information to guide the robotic device participant 708 to a break area that has cups and a soda machine rather than a different break area that only offers soda cans via a cooling unit such as a refrigerator.

In addition to issuing an initial instruction 724, the AI agent participant 706 and the robotic device participant 708 can implement customized exchanges 802 until the task is completed. This enables autonomous control of the robotic device participant 708 by the AI agent participant 706. Continuing the specific example above (again), the robotic device participant 708 may report a status update back to the AI agent participant 706 as part of the customized exchanges 802. The status update may indicate that after looking, the robotic device participant 708 has determined that a particular break area is temporarily out of Joe's favorite flavor of soda. In response, the AI agent participant 706 can automatically (without involving Joe) generate and transmit an updated instruction to the robotic device participant 708 as part of the customized exchanges 802. The updated instruction may command the robotic device participant 708 to retrieve and deliver a different flavor of soda (e.g., Joe's second favorite flavor) or to visit a different break area to retrieve and deliver Joe's favorite flavor of soda. Consequently, a combination of the AI agent participant 706 and the robotic device participant 708 can perform some or all of the decisions and/or functions a human participant would typically perform to complete a mission 110 in a geographical environment 112.

As described above with respect to FIG. 3, the AI model 718 may be a primary AI model 320 that implements general intelligence support for the interaction environment 114 or a secondary AI model 324 that is dedicated to support tasks capable of being performed by the robotic device participant 708. In one example, the primary AI model serves as a conduit between humans and secondary AI model(s) 324. Accordingly, FIG. 8 illustrates that the AI agent participant 706 can be a general AI support agent 808 for different types of robotic devices 810, and therefore, the general AI support agent 808 may be required to obtain capability data 806 for the robotic device participant 708.

Alternatively, FIG. 8 illustrates that the AI agent participant 706 can be a dedicated AI support agent 812 for a specific type of robotic device 814 that may not be required to obtain the capability data 806 for the robotic device participant 708 as it is already configured and/or trained to only support one specific type of robotic device. In this scenario, the collaboration session 102 may automatically invite and/or add the AI agent participant 706 in response to the robotic device participant 708 joining the collaboration session 102. For instance, a primary AI model 320 may be used to determine robotic devices that have been added to a collaboration session 102 and then automatically invite and/or add secondary AI models 324, or the dedicated AI support agents 812, that are already familiar with the robotic devices that have been added to the collaboration session 102.

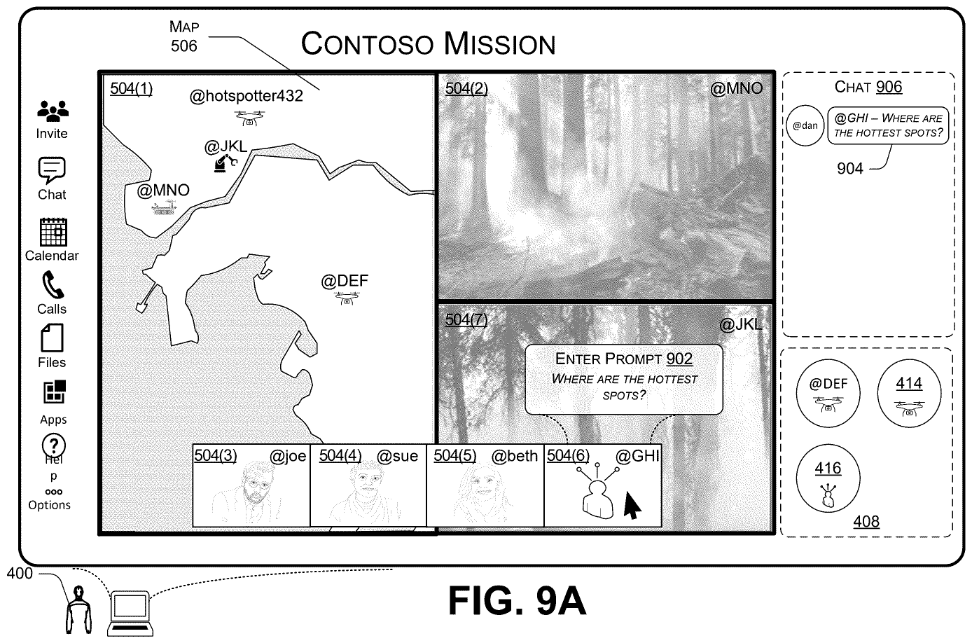

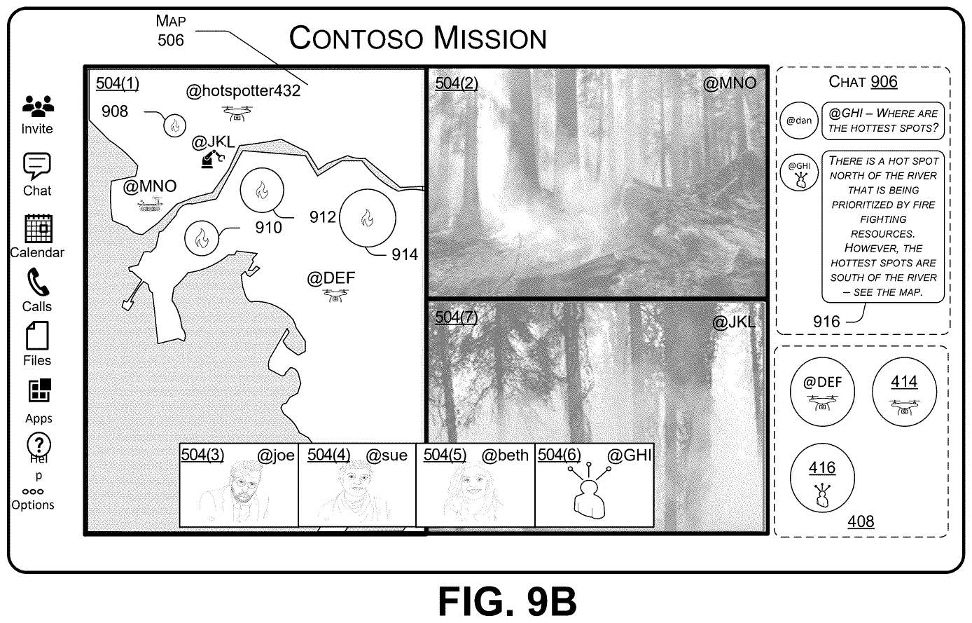

FIG. 9A illustrates an example interaction environment that exposes a mechanism for a human participant 400 (e.g. “@dan”) to provide input (e.g., a prompt, a chat message) that causes an artificial intelligence agent to perform an analysis 720 on an aspect of the interaction environment. The example interaction environment of FIG. 9A is similar to that shown in FIG. 5D. In this example, the mechanism 710 to provide an input 704 is either a direct prompt entry 902 via a graphical representation 116 associated with an AI agent participant (e.g., AI agent participant “@GHI”) or a chat message 904 entered via a chat 906 associated with the collaboration session 102 and interaction environment.

As shown, the input 704 states “Where are the hotspots?” When the input 704 is entered as a chat message 904 via a more general chat 906 associated with the collaboration session 102, the input 704 may need to explicitly identify the AI agent participant (e.g., “@GHI”) to which the input 704 is directed, provided a scenario where there is more than one AI agent participant in the collaboration session 102. Alternatively, a general AI support agent 808 may be able to infer that the input 704 is directed to a particular type of robotic device participant based on a type of analysis and redirect the input 704 to a dedicated AI support agent 812 that supports the particular type of robotic device participant.

When the input 704 is entered via the direct prompt entry 902 via the graphical representation 116 associated with the AI agent participant (e.g., by right clicking on the area 504(6) in which AI agent participant “@GHI” is displayed), the input 704 does not need to explicitly identify the AI agent participant to which the input 704 is directed.

FIG. 9B illustrates an example of how the analysis 720 by the AI agent participant can be displayed as AI data 722. In one example, the AI agent participant “@GHI” can overlay their analysis 720 on a map 506. Here, the AI agent participant “@GHI” has analyzed the video streams and/or other types of sensor data (e.g., smoke data, thermal data) and determined locations in the geographical environment 112, represented by icons 908, 910, 912, 914 on the map 506, that have the hottest spots (e.g., locations where a fire is burning the strongest). Moreover, the AI agent participant “@GHI” graphically indicates the strength of the heat, or fire, based on a size of the circle around the fire icons. Thus, AI agent participant “@GHI” has determined that the location represented by icon 908 (e.g., where most of the fire fighting resources are shown to be deployed) is not as strong as the heat at the location represented by icon 914, for example.

Additionally, the AI agent participant “@GHI” can respond in the chat 906 with its own chat message 916 describing the analysis 720—“There is a hot spot north of the river that is being prioritized by fire fighting resources. However, the hottest spots are south of the river - see the map.” Accordingly, via the collaboration session 102 and interaction environment 114, the human participants 104(1-N) can better understand what is happening in the geographical environment 112 so they can make decisions and/or engage (e.g., call on) various resources (e.g., robotic device participants 106(1-N), AI agent participants 108(1-N)) to assist with mission 110.

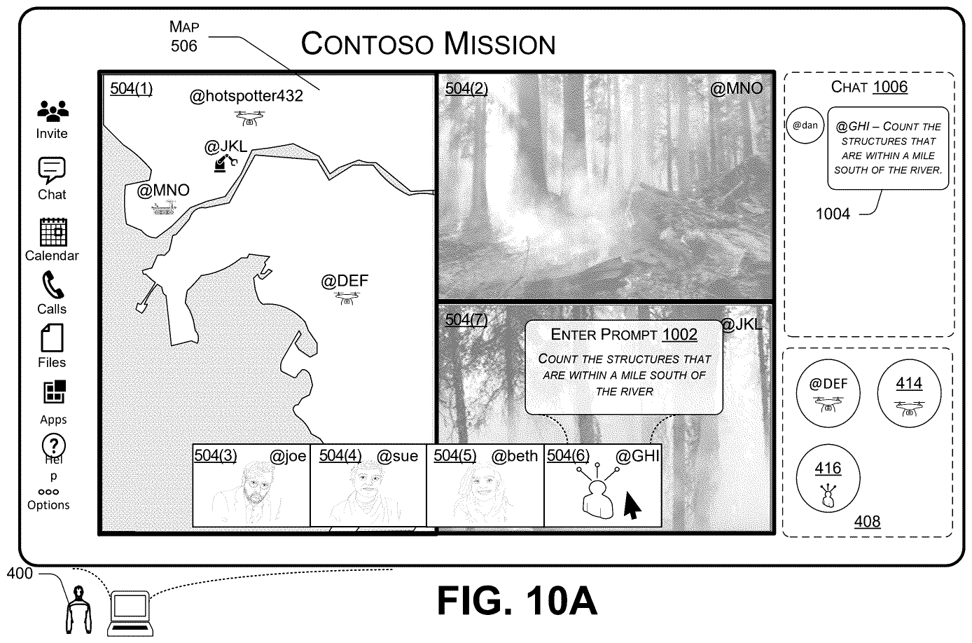

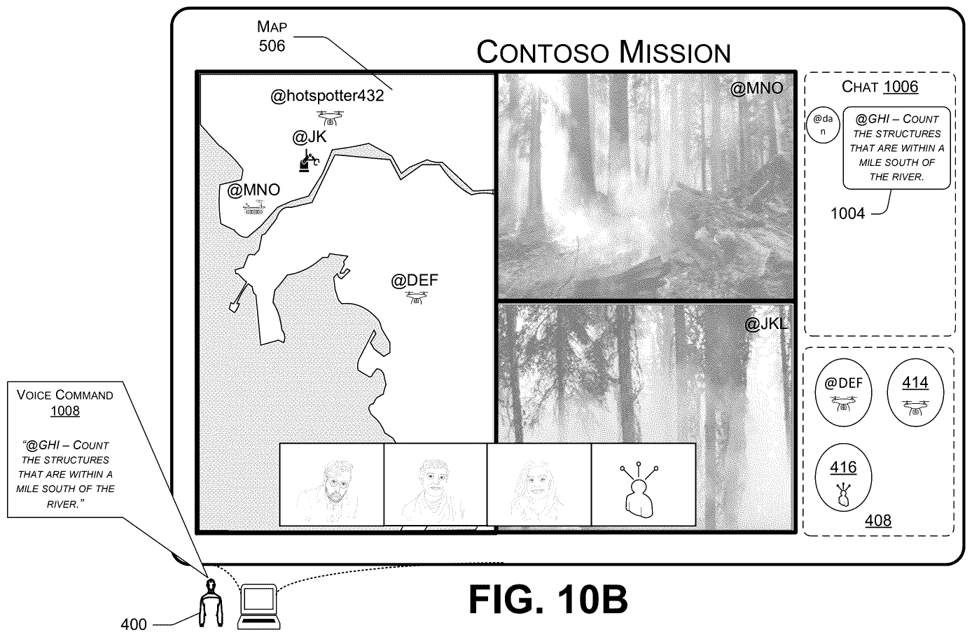

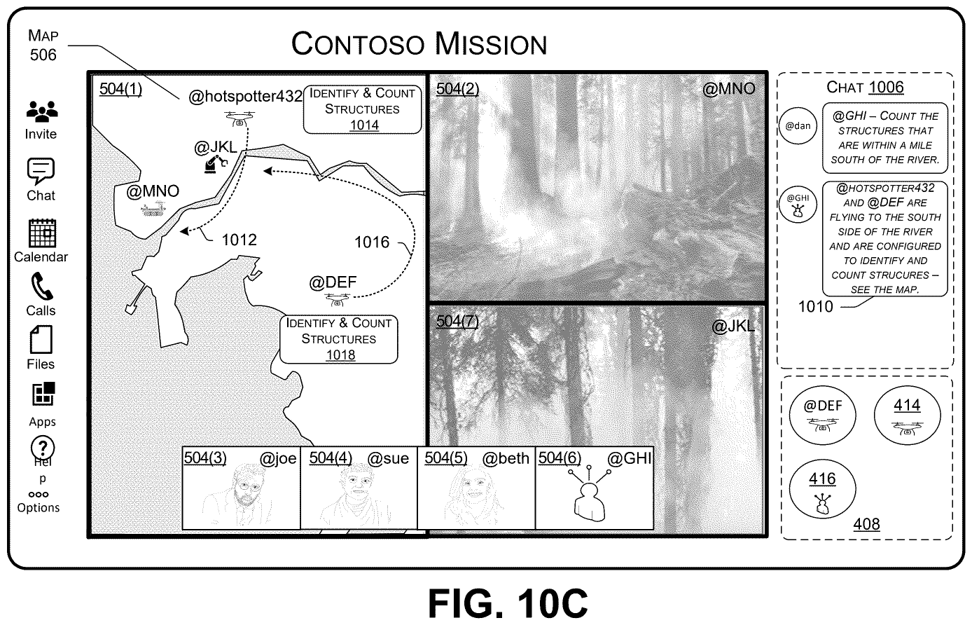

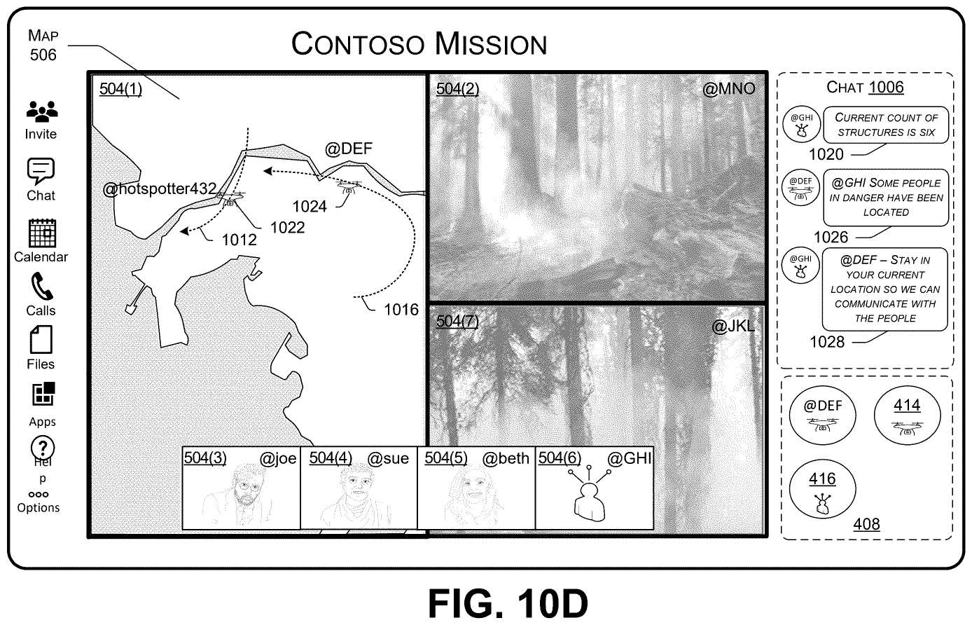

FIG. 10A illustrates an example interaction environment that exposes a mechanism for a human participant 400 (e.g. “@dan”) to provide input (e.g., a text-based prompt, a chat message) that causes an artificial intelligence agent to generate an instruction 724 for a robotic device participant to execute a task 712 associated with a mission 110 in a geographical environment 112. The example interaction environment of FIG. 10A is again similar to that shown in FIG. 5D. In this example, the mechanism 710 to provide an input 704 is either a direct prompt entry 1002 via a graphical representation 116 associated with an AI agent participant (e.g., AI agent participant “@GHI”) or a chat message 1004 entered via a chat 1006 associated with the collaboration session 102 and interaction environment.

As shown, the input 704 states “Count the structures that are within a mile south of the river.” When the input 704 is entered as a chat message 1004 via a more general chat 1006, the input 704 may need to explicitly identify the AI agent participant (e.g., “@GHI”) to which the input 704 is directed, provided a scenario where there is more than one AI agent participant in the collaboration session 102. Alternatively, a general AI support agent 808 may be able to infer that the input 704 is directed to a particular type of robotic device participant based on a type of task that is described and redirect the input 704 to a dedicated AI support agent 812 that supports the particular type of robotic device participant.

When the input 704 is entered as the direct prompt entry 1002 via the graphical representation 116 associated with the AI agent participant (e.g., by right clicking on the area 504(6) in which AI agent participant “@GHI” is displayed), the input 704 does not need to explicitly identify the AI agent participant to which the input 704 is directed.

FIG. 10B illustrates how the human participant 400 can provide the input 704 via a voice-based command 1008 that is spoken aloud—“@GHI—Count the structures that are within a mile south of the river”—and that is audibly recognized and processed as the input 704 by the collaboration session 102. Based on the example input 704 described with respect to FIGS. 10A and/or 10B, AI agent participant “@GHI” generates and transmits an instruction 724 for a robotic device participant to execute a task described via the input 704. The instruction 724 includes text and/or executable code that is transmitted in a format that can be understood by the robotic device participant.

As described herein with respect to FIG. 10C, AI agent participant “@GHI” transmits the instruction 724 to the robotic device participant “@hotspotter432” and to the robotic device participant “@DEF”. Furthermore, FIG. 10C illustrates an example of how the status of the task 726 being executed by the robotic device participants “@hotspotter432” and “@DEF” (both UAVs) can be displayed in the interaction environment. In one example, AI agent participant “@GHI” communicates the status update(s) via the chat 1006 in its own chat message. A first chat message 1010 states—“@hotspotter432 and @DEF are flying to the south side of the river and are configured to identify and count structures—see the map.”

Additionally or alternatively, the status update(s) can be displayed via the map 506 of the geographical environment. As shown in FIG. 10C, AI agent participant “@GHI” has transmitted an instruction 724 for “@hotspotter432” to fly on a path headed southwest along the river. The path is graphically shown one the map 506 via the dotted arrow 1012. Also graphically shown is an indicator that “@hotspotter432” is currently identifying and counting count structures 1014. Moreover, as shown in FIG. 10C, AI agent participant “@GHI” has transmitted an instruction 724 for “@DEF” to fly on a different path that first heads north and then heads due west along the river. This different path is graphically shown on the map 506 via the dotted arrow 1016. Also graphically shown is an indicator that “@DEF” is currently identifying and counting count structures 1018. Consequently, the AI agent participant “@GHI” has coordinated the movement of two different UAVs in order to efficiently and effectively execute the task—“Count the structures that are within a mile south of the river.” Furthermore, prior to issuing the instructions 724 to fly along the paths 1012 and 1016, the AI agent participant “@GHI” may have needed to first outline a zone that contains a mile of land inland from the river on the south side.

FIG. 10D illustrates another example of how the status of the task being executed by the robotic device participant by can be displayed in the interaction environment and how autonomous control of the robotic device participant can be implemented by the artificial intelligence agent participant. As illustrated, the chat 1006 includes a message 1020 that states—n that the “current count of structures is six.” This comes at a later time as the UAVs “@hotspotter432” and “@DEF” have flown along the paths 1012 and 1016 to the respective locations 1022 and 1024. In addition to finding and counting structures, “@DEF” has communicated back some data indicating that it has located some people in danger (e.g., the chat includes a message 1026 stating—“@GHI Some people in danger have been located.”). In response, “@GHI” can autonomously control “@DEF” by issuing an updated instruction to “Stay in your current location so we can communicate with the people”, as captured in message 1028. Then, “@GHI” may open a communication channel for the human participants (e.g., “@dan”, “@joe”, “@sue”, “@beth”) to talk with the people in danger via equipment configured on the robotic device participant “@DEF”.

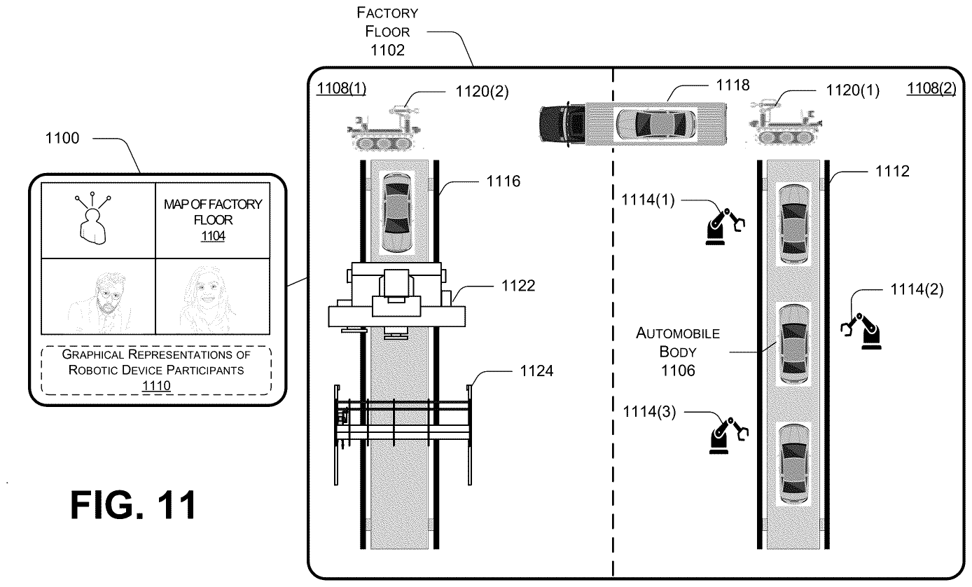

FIG. 11 illustrates another example context for an interaction environment 1100 where graphical representations for at least one human participant, at least one robotic device participant, and at least one artificial intelligence agent participant are displayed. The context in FIG. 11 relates to a factory floor 1102. Accordingly, the interaction environment 1100 includes a map of the factory floor 1104 in an area designated as a primary area.

In this example, the factory floor 1102 is set up to manufacture an automobile body 1106 using different types of robotic devices. Moreover, the factory floor 1102 is divided into two areas 1108(1) and 1108(2). Each of the robotic devices in FIG. 11 are participants in the collaboration session 102, and therefore, are graphically represented in the interaction environment 1100 (e.g., via secondary area 1110).

Area 1108(2) includes a first moving belt 1112 so that arm-based robotic device participants 1114(1-3) can place and attach a particular part (e.g., a bumper, a windshield) to the automobile body 1106. Once the arm-based robotic device participants 1114(1-3) place and attach the particular part to the automobile body 1106, the automobile body 1106 needs to be moved from the first moving belt 1112 in area 1108(2) to a second moving belt 1116 in area 1108(1). Accordingly, the factory floor 1102 includes a transporter robotic device participant 1118 as well as two loader/unloader robotic device participants 1120(1-2). That is, a first loader/unloader robotic device participant 1120(1) loads the automobile body 1106 from the first moving belt 1112 on to the transporter robotic device participant 1118 and a second loader/unloader robotic device participant 1120(2) unloads the automobile body 1106 from the transporter robotic device participant 1118 on to the second moving belt 1116.

The second moving belt 1116 in area 1108(1) of the factory floor 1102 is configured to paint the automobile body 1106. Accordingly, a first type of applier robotic device participant 1122 is tasked with applying the primer to the automobile body 1106 and then a second type of applier robotic device participant 1124 is tasked with applying different colors of paint to the automobile body 1106. Consequently, via the collaboration session 102 and interaction environment 1100, humans, robotic devices, and AI agents can coordinate to efficiently and effectively complete a mission 110 related to preparing an automobile body 1106 in the geographical environment 112 of a factory floor 1102.

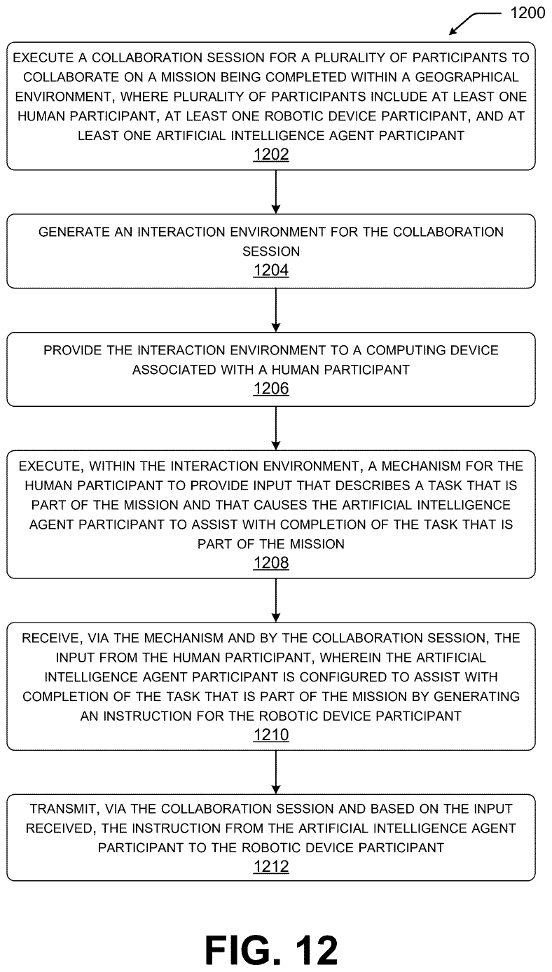

FIG. 12 is a flowchart depicting an example process 1200 for executing a collaboration session that enables a human participant to provide input that causes an artificial intelligence agent participant to generate and transmit an instruction to a robotic device participant. The process 1200 begins at operation 1202 where a system executes a collaboration session for a plurality of participants to collaborate on a mission being completed within a geographical environment. As described above, the plurality of participants includes at least one human participant, at least one robotic device participant, and at last one artificial intelligence agent participant.

At operation 1204, the system generates an interaction environment for the collaboration session.

At operation 1206, the system provides the interaction environment to a computing device associated with the human participant.

At operation 1208, the system executes, within the interaction environment, a mechanism for the human participant to provide input that describes a task that is part of the mission and that causes the artificial intelligence agent participant to assist with completion of the task that is part of the mission.

At operation 1210, the system receives, via the mechanism and by the collaboration session, the input from the human participant. As described above, the artificial intelligence agent participant is configured to assist with completion of the task that is part of the mission by generating an instruction for the robotic device participant.

At operation 1212, the system transmits, via the collaboration session and based on the input received, the instruction from the artificial intelligence agent participant to the robotic device participant.

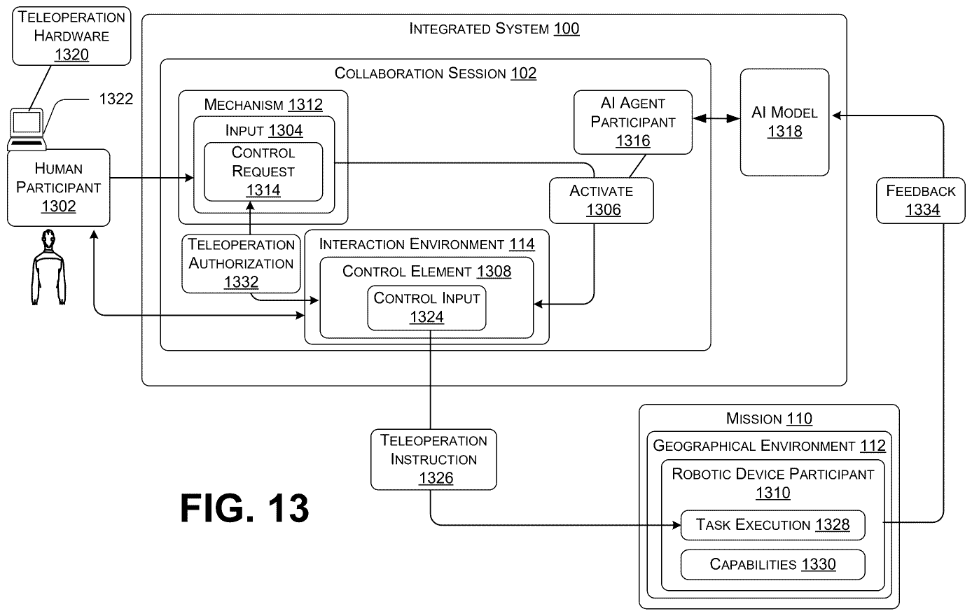

FIG. 13 illustrates further aspects of the integrated system 100 executing the collaboration session 102, as described above with respect to FIGS. 1 and 3. FIG. 13 is directed to enabling a human participant 1302 (e.g., one of human participants 104(1-N)) to provide input 1304 that causes the collaboration session 102 to activate 1306 a control element 1308 within the interaction environment 114. The control element 1308 is associated with a robotic device participant 1310 (e.g., one of robotic device participants 106(1-N)) that is deployed to a geographical environment 112 to assist with completion of a mission 110.

In one example, via execution of the collaboration session 102, the integrated system 100 exposes a mechanism 1312 for the human participant 1302 to provide the input 1304. As further described herein, the mechanism 1312 is configured to receive GUI-based selection inputs, text-based inputs, sensor-based inputs, and/or voice inputs. The input 1304 can serve as a control request 1314 for the human participant 1302, which thereby communicates an intent of the human participant 1302 to remotely control, or “teleoperate”, the robotic device participant 1310 through the collaboration session 102.

Accordingly, the mechanism 1312 receives the input 1304 from the human participant 1302 in this example and the input 1304 causes the collaboration session 102 to activate 1306 the control element 1308 in the context of the interaction environment 114. In various examples further described herein, the input 1304 triggers an AI agent participant 1316 (and corresponding AI model 1318) to activate 1306 the control element 1308.

Alternatively, the AI agent participant 1316 can activate 1306 the control element 1308 on its own without the input 1304 from the human participant 1302. For instance, the AI agent participant 1316 and/or the robotic device participant 1310 can determine that a current situation in the geographical environment 112 requires human assistance, and accordingly, can activate 1306 the control element 1308 automatically without the input 1304 from the human participant 1302.

In further examples, the control element 1308 is activated 1306 automatically in response to the robotic device participant 1310 joining the collaboration session 102. Consequently, the control element 1308 can be persistently displayed and/or configured for use while the robotic device participant 1310 is participating in the collaboration session 102 or when the graphical representation 116 for the robotic device participant 1310 is displayed in an area of the interaction environment 114 that is designated as primary.

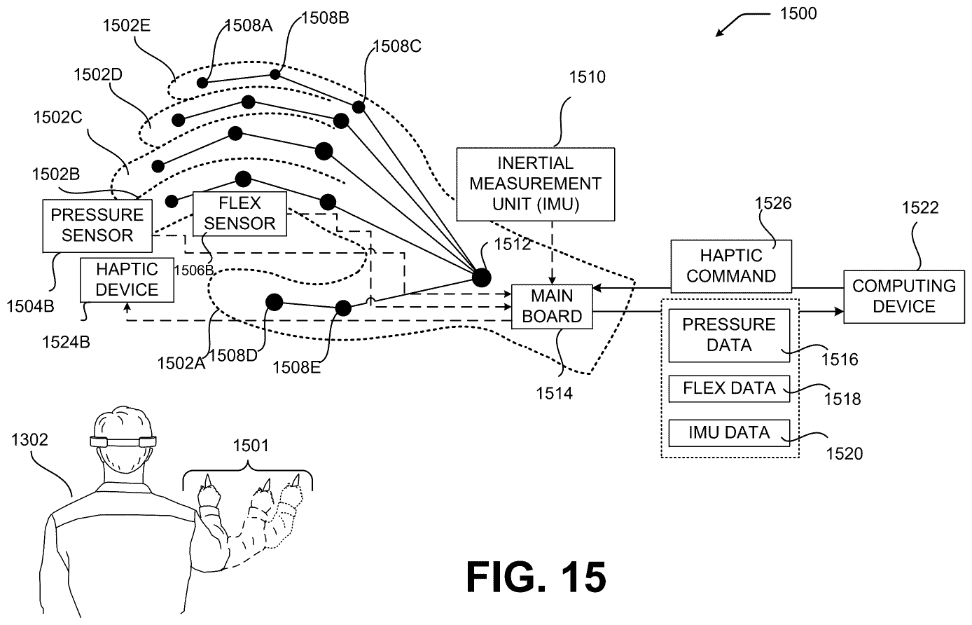

In one example, activating 1306 the control element 1308 includes displaying the control element 1308 within the interaction environment 114. Accordingly, the control element 1308 may be a virtual control element that operates based on a selection (e.g., a touch-based selection, a mouse-based selection, a gesture-based selection). In another example, activating 1306 the control element 1308 includes configuring the interaction environment 114 to engage with teleoperation hardware 1320 that is configured in association with a computing device 1322 being used by the human participant 1302 to participate in the collaboration session 102. Therefore, the control element 1308 can be functionality that supports the teleoperation hardware 1320. The teleoperation hardware 1320 can include a physical joystick, a physical keyboard, a sensory form factor device (e.g., a “rig”) that overlays a human body or part of a human body (an example of which is described below with respect to FIG. 15), or other types of physical control devices capable of providing a control input 1324.

Consequently, the collaboration session 102 is configured to receive, via a displayed control element 1308 or a control element 1308 implementing functionality to act on input from the teleoperation hardware 1320, the control input 1324 from the human participant 1302. Based on the control input 1324, the collaboration session 102 is configured to transmit a teleoperation instruction 1326 to the robotic device participant 1310. The teleoperation instruction 1326 causes the robotic device participant 1310 to execute a task 1328 (e.g., pick an object up, move in a certain direction, change a speed at which the robotic device participant 1310 is moving, drop an object off, attach a widget). In various examples, the teleoperation instruction 1326 is generated and transmitted via a file that includes text and/or executable code in a format that is understood by the robotic device participant 1310 such that the robotic device participant 1310 can execute the task 1328.

In one example further described herein, the control element 1308 is a universal control element that is common to different types of robotic device participants. The universal control element can be mapped to a task that is common to the different types of robotic device participants. For instance, employing a virtual joystick to indicate movement to the right transmits a teleoperation 1326 that causes each of the different types of robotic device participants to move to the right. Alternatively, the universal control element can be mapped to different tasks that are respectively associated with different types of robotic device participants. For instance, a selection of the control element for a first type of robotic device participant can cause the first type of robotic device participant to push an object, while a selection of the same control element for a second type of robotic device participant can cause the second type of robotic device participant to grab and pull an object. Thus, the tasks 1328 that are executed by a teleoperation instruction 1326 transmitted based on a control input 1324 to a universal control element may depend on the capabilities 1330 of the robotic device participant 1310, as described above.

In another example further described herein, the control element 1308 is a customized control element for a specific type of robotic device participant. Accordingly, the AI agent participant 1316 (e.g., a dedicated AI support agent 812) may be configured to identify and activate 1306 the customized control element for the specific type of robotic device participant. Thus, the tasks 1328 that are executed by a teleoperation instruction 1326 transmitted based on a control input 1324 to a customized control element may depend on the capabilities 1330 of the robotic device participant 1310, as described above.

In various examples, the collaboration session 102 is configured to check whether the human participant 1302 has teleoperation authorization 1332 to teleoperate the robotic device participant 1310. As described above, the collaboration session 102 enables a collaborative approach to performing tasks to complete the mission 110 in the geographical environment 112. Accordingly, more than one human participant 1302 may teleoperate a robotic device participant 1310 in the context of the collaboration session 102. Further, control of the robotic device participant 1310 for teleoperation purposes may be passed from one human participant 1302 to another human participant in the context of the collaboration session 102. As many robotic devices are configured to perform more complex and/or dangerous tasks, the teleoperation may need to be authorized to ensure safety to the people in the geographical environment 112 and/or to prevent damage to objects surrounding the robotic device participant 1310 in the geographical environment 112. For instance, the teleoperation authorization 1332 may ensure the human participant 1302 has enough experience to teleoperate the robotic device participant 1310 (e.g., via checking a user profile or account for certifications indicating required trainings have been completed or a threshold number of training-based operating hours has been satisfied).

In additional examples, the robotic device participant 1310 provides feedback 1334 to the AI model 1318 describing the task execution 1328 based on the teleoperation instruction 1326. The feedback 1334 is able to train and fine-tune the AI model 1318 (e.g., a LLM, a SLM, a LAM, a SAM) so that more accurate and informed task automation can be implemented by the AI agent participant 1316. For instance, if the teleoperation instruction 1326 halts forward movement of a UAV due to high winds and a risk the control of the UAV may be lost (which can lead to a crash), the feedback 1334 can note the wind speed being sensed by the UAV (and being viewed by the human participant 1302) at the time the teleoperation instruction 1326 is received. Accordingly, the AI model 1318 can ensure that future automated control of the UAV by the AI agent participant 1316 does not instruct the UAV to continue to fly into a zone that has wind speeds higher than the wind speed noted in the feedback 1334.

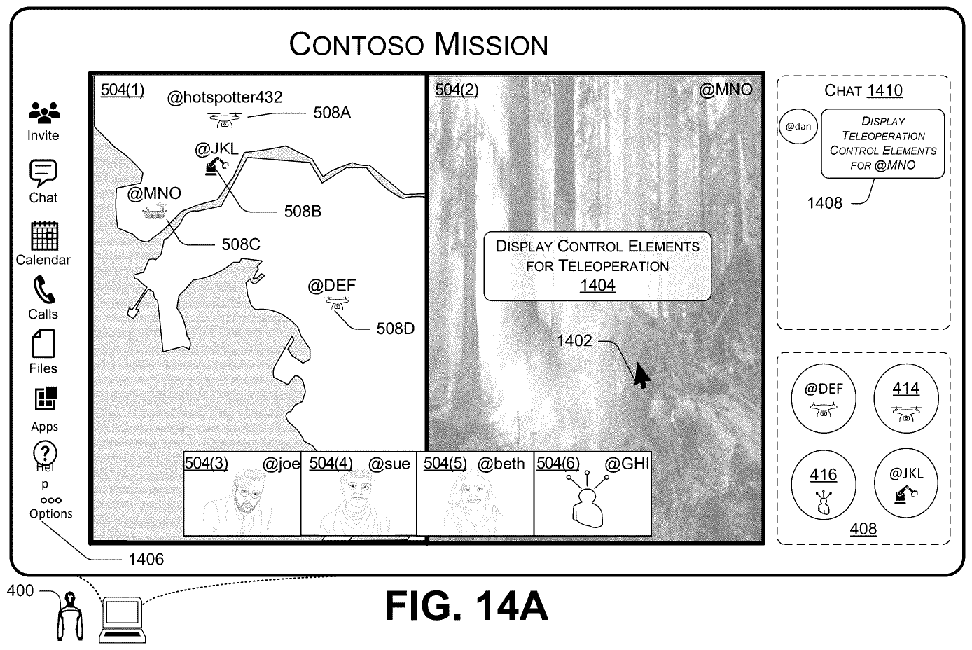

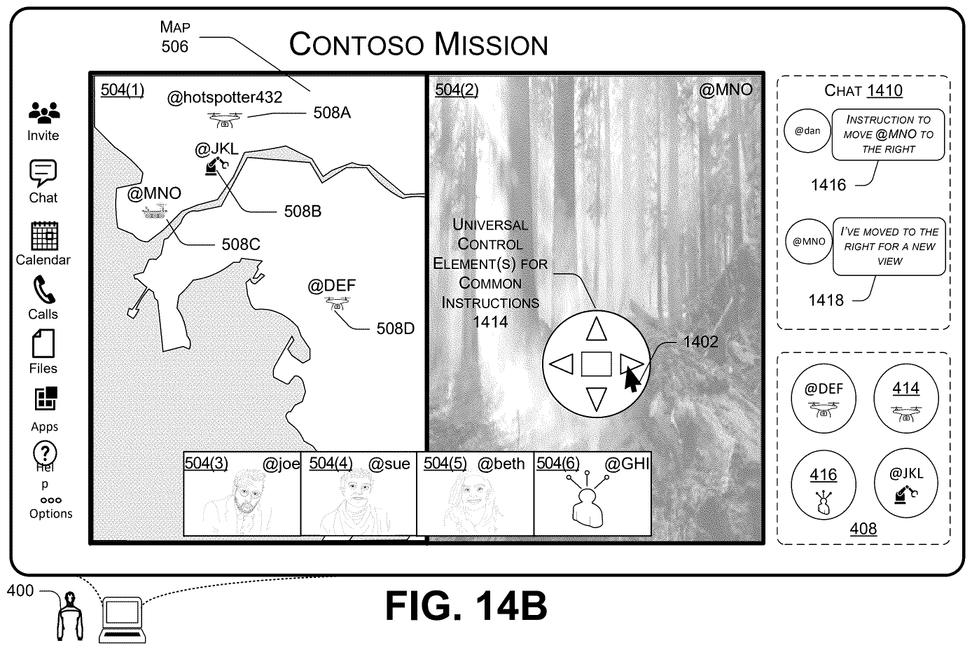

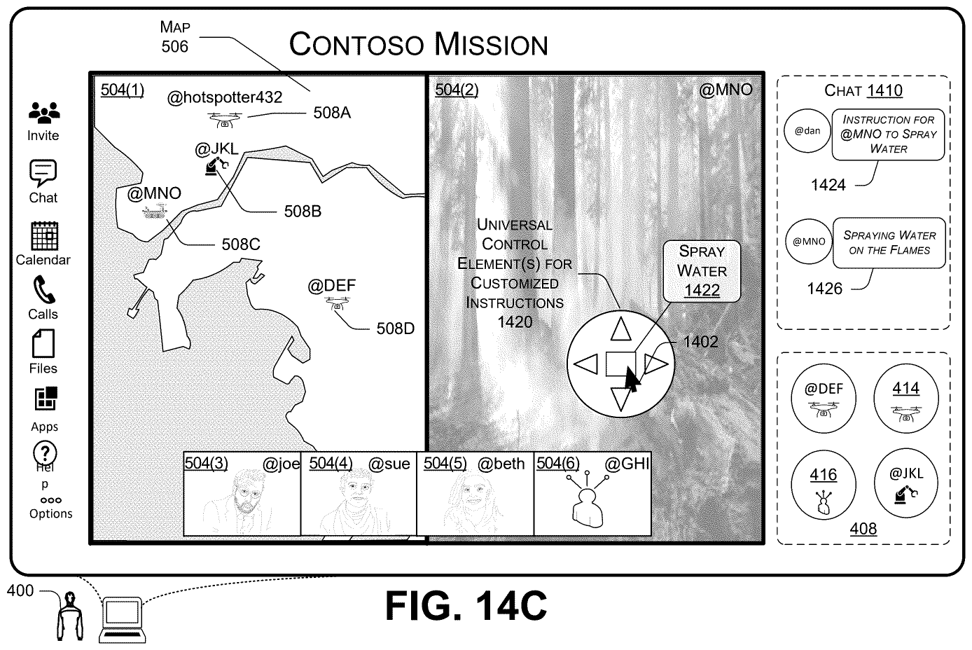

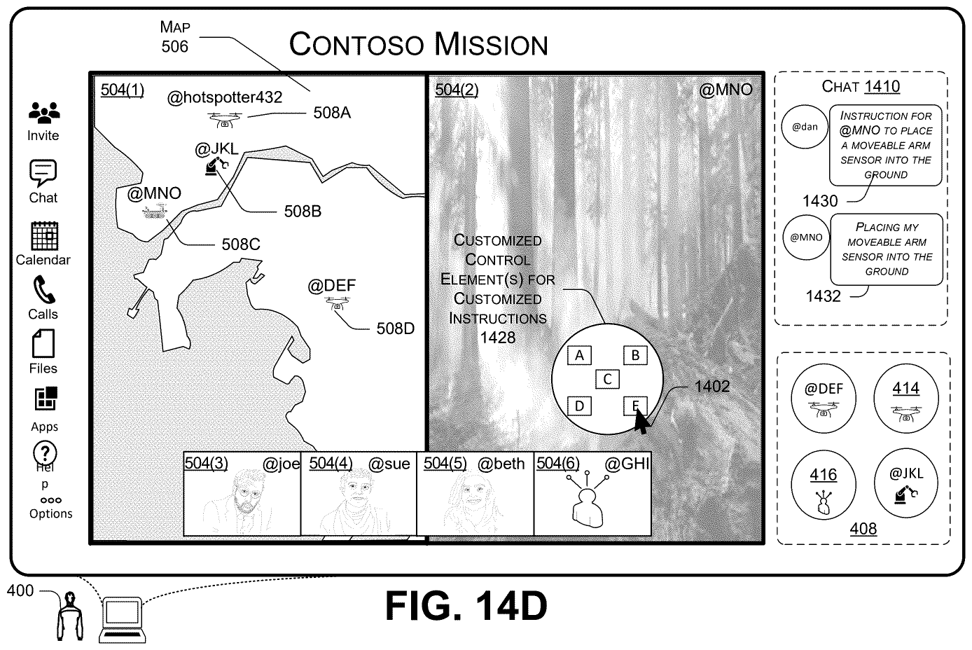

FIG. 14A illustrates an example interaction environment that exposes a mechanism for a human participant 400 (e.g. “@dan”) to provide input that causes the interaction environment to activate 1306 (e.g., display) a control element 1308 (described below with respect to FIGS. 14B-D) for the robotic device participant “@MNO”. The example interaction environment of FIG. 14A is similar to that shown in FIG. 5C. In one example, the mechanism 1312 for the human participant 400 to provide input 1304 that activates 1306 the control element 1308 comprises right clicking on the area 504(2) associated with robotic device participant “@MNO” using a user-directed input mechanism 1402 (e.g., a mouse and/or cursor) and selecting a menu option to display control elements for teleoperation 1404. In another example, the mechanism 1312 for the human participant 400 to provide input 1304 that activates 1306 the control element 1308 comprises selecting a menu of options 1406 using the user-directed input mechanism 1402 and selecting the menu option to display control elements for teleoperation 1404. In yet another example, the mechanism 1312 for the human participant 400 to provide input 1304 that activates 1306 the control element 1308 comprises entering a message 1408 into a chat 1410. The message 1408 identifies the robotic device participant “@MNO” and requests that the collaboration session “Display teleoperation control elements for @MNO”.

As an alternative to the examples of the inputs 1304 that cause activation 1306 of the control element 1308 discussed in FIG. 14A, the AI agent participant 1316 can activate 1306 the control element 1308 on its own without the input 1304. For instance, the AI agent participant 1316 and/or the robotic device participant 1310 can determine that a current situation in the geographical environment 112 requires human assistance, and accordingly, can activate 1306 (display) the control element 1308 (examples of which are discussed below with respect to FIGS. 14-B-D) automatically without the input 1304 from the human participant 1302.

In further examples, the control element 1308 is activated 1306 automatically in response to the robotic device participant 1310 joining the collaboration session 102. Consequently, the control element 1308 (examples of which are discussed below with respect to FIGS. 14-B-D) can be persistently displayed and/or configured for use while the robotic device participant 1310 is participating in the collaboration session 102 or when the graphical representation 116 for the robotic device participant 1310 is displayed in an area of the interaction environment 114 that is designated as primary.

FIG. 14B illustrates an example interaction environment that displays universal control element(s) 1414 (e.g., a virtual joystick) configured to receive control input that causes a common teleoperation instruction 1326 to be transmitted to different types of robotic device participants. Here a selection of the up arrow displayed via the universal control element(s) 1414 causes the robotic device participant “@MNO” to move forward, and the same selection of the up arrow would cause other types of robotic device participants to move forward as well. A selection of the down arrow displayed via the universal control element(s) 1414 causes the robotic device participant “@MNO” to move backward, and the same selection of the down arrow would cause other types of robotic device participants to move backward as well. A selection of the right arrow displayed via the universal control element(s) 1414 causes the robotic device participant “@MNO” to move to the right, and the same selection of the right arrow would cause other types of robotic device participants to move to the right as well. A selection of the left arrow displayed via the universal control element(s) 1414 causes the robotic device participant “@MNO” to move to the left, and the same selection of the left arrow would cause other types of robotic device participants to move left as well. Finally, a selection of the center button displayed via the universal control element(s) 1414 causes the robotic device participant “@MNO” to put on brakes and/or halt movement, and the same selection of center button would cause other types of robotic device participants to put on brakes and/or halt movement. Other universal control elements may control elevation for different types of UAVs (e.g., increase elevation or decrease elevation).

The video feed shown in area 504(2) may provide visual status updates, to the human participant 1302, related to the task execution 1328 and the teleoperation instruction 1326. In additional examples, FIG. 14B illustrates how status updates associated with the task execution 1328 and the teleoperation instruction 1326 can be reflected in the chat 1410. As shown, “@dan” provided input via the user-directed input mechanism 1402 on the right arrow, and this causes an “Instruction to move @MNO to the right”, as shown in chat message 1416. After or while executing the task, the robotic device participant “@MNO” reports back with a chat message 1418 indicating that “I've moved to the right for a new view”.

FIG. 14C illustrates an example interaction environment that displays universal control element(s) 1420 (e.g., a virtual joystick) configured to receive a control input that causes a customized teleoperation instruction to be transmitted to a specific type of robotic device participant. In the example of FIG. 14C, while the directional arrows may cause common teleoperation instructions, the center button displayed via the universal control element(s) 1420 can be mapped to different tasks that are respectively associated with different types of robotic device participants. For instance, a selection of the center button for the robotic device participant “@MNO” can cause the robotic device participant “@MNO” to spray water. In contrast, a selection of the same center button for the robotic device participant “@JKL” can cause the robotic device participant “@JKL” to move dirt. In various examples, guidance as to which teleoperation instruction the control element is associated with can be provided via the interaction environment, as shown via user interface element 1422.

In the example of FIG. 14C, the status updates are also reflected in the chat 1410. As shown, “@dan” provided input via the user-directed input mechanism 1402 on the center button, and this causes an “Instruction for @MNO to spray water”, as shown in chat message 1424. After or while executing the task, the robotic device participant “@MNO” reports back with a chat message 1426 indicating that “Spraying water on the flames”.

FIG. 14D illustrates an example interaction environment that displays customized control element(s) 1428 (e.g., a virtual joystick) configured to receive control input that causes a customized teleoperation instruction to be transmitted to a specific type of robotic device participant. In the example of FIG. 14D, a selection of button associated with task “A” displayed via the customized control element(s) 1428 causes the robotic device participant “@MNO” to rotate the camera to the left. A selection of button associated with task “B” displayed via the customized control element(s) 1428 causes the robotic device participant “@MNO” to rotate the camera to the right. A selection of button associated with task “C” displayed via the customized control element(s) 1428 causes the robotic device participant “@MNO” to capture a still image using the camera. A selection of button associated with task “D” displayed via the customized control element(s) 1428 causes the robotic device participant “@MNO” to spray water. Finally, a selection of button associated with task “E” displayed via the customized control element(s) 1428 causes the robotic device participant “@MNO” to place a moveable arm sensor into the ground to obtain soil data.

In various examples, the customized control element(s) 1428 are configured for a specific set of capabilities 1330 of the robotic device participant “@MNO”. Accordingly, the AI agent participant 1316 (e.g., a dedicated AI support agent 812) may be configured to identify and activate 1306 the customized control element(s) 1428 for the specific type of robotic device participant.

In the example of FIG. 14D, the status updates are also reflected in the chat 1410. As shown, “@dan” provided input via the user-directed input mechanism 1402 on the button associated with task “E”, and this causes an “Instruction for @MNO to place a moveable arm sensor into the ground”, as shown in chat message 1430. In response, the robotic device participant “@MNO” reports back with a chat message 1432 indicating that its “Placing my moveable arm sensor into the ground”.

FIG. 15 illustrates an example piece of teleoperation hardware 1320 that is a sensory form factor device in the shape of a digital glove 1500. As described above, teleoperation hardware 1320 such as digital glove 1500 can engage with, or provide control inputs 1324 to, an activated control element 1308 configured in the interaction environment 114. While the digital glove 1500 is shown and discussed herein, it is understood that other sensory form factor devices can be used to sense and/or communicate movements and/or gestures (an example of which is shown via 1501) by human participant 1302 as control inputs 1324 to the control element 1308 (e.g., the human participant 1302 can be fitted with an upper body or a full body “rig”).

The digital glove 1500 is configured with sensors to detect the pose of the wearer's hand and pressure exerted at the fingertips of the wearer's hand. For instance, the fingers 1502A-E of the digital glove 1500 can be equipped with flex sensors, also called “bend” sensors, capable of detecting the amount of flex or bend in a wearer's fingers. For instance, the fingers 1502A-E of the digital glove 1500 can be equipped with sensors based upon capacitive/piezoresistive sensing. In the example configuration shown in FIG. 15, only a single flex sensor 1506B has been illustrated in the index finger 1502B of the digital glove 1500 for ease of reference. It is to be appreciated, however, that the digital glove 1500 can be configured with one or more flex sensors 1506 in each of the fingers 1502A-E.

The flex sensors 1506 can be mounted in the digital glove 1500 such that the flex of the joints of a wearer's hand can be measured. For example, the digital glove 1500 can include flex sensors 1506 for measuring the flex in a wearer's distal interphalangeal (“DIP”) joint 1508A, proximal interphalangeal (“PIP”) joint 1508B, metacarpophalangeal (“MCP”) joint 1508C, interphalangeal (“IP”) joint 1508D, and/or metacarpophalangeal (“MCP”) joint 1508E.

Tactile pressure sensors 1504 (which might be referred to herein as “pressure sensors 1504”) can also be mounted in the fingertips of the digital glove 1500 to sense the amount of pressure exerted by the fingertips of a wearer. In the example configuration shown in FIG. 15, only a single pressure sensor 1504B has been illustrated in the tip of the index finger 1502B of the digital glove 1500 for ease of reference. It is to be appreciated, however, that the digital glove 1500 can be configured with one or more pressure sensors 1504 in the tips of each of the fingers 1502A-1502E. Pressure sensors can be mounted at other positions in the digital glove 1500 in other configurations.

The digital glove 1500 might also include an inertial measurement unit (“IMU”) 1510. The IMU 1510 can detect the pronation and supination of the wearer's hand. The IMU 1510 might be mounted in the digital glove 1500 at a location at or around the wearer's wrist 1512. The digital glove 1500 can also, or alternately, include other types of sensors in order to detect other aspects of the pose of a wearer's hand.

The digital glove 1500 can also include output devices, such as one or more haptic devices 1524B, to provide feedback to a wearer. In the example configuration shown in FIG. 15, only a single haptic device 1524B has been illustrated in the tip of the index finger 1502B of the digital glove 1500 for ease of reference. It is to be appreciated, however, that the digital glove 1500 can be configured with one or more haptic devices 1524 in the tips of each of the fingers 102A-102E. Haptic devices 1524 can be mounted at other positions in the digital glove 1500 in other configurations. The haptic devices 1524 can be implemented using various technologies such as, but not limited to, linear resonant Actuator (LRA), eccentric rotating mass (ERM), voice-coil, and various other types of actuating hardware.

As illustrated in FIG. 15, the digital glove 1500 is also equipped with a main board 1514. The main board 1514 is a circuit board that receives pressure data 1516 describing the pressure exerted by a wearer's fingers from the pressure sensors 1504. The main board 1514 also receives flex data 1518 describing the flex in a wearer's fingers from the flex sensors 1506. The main board 1514 also receives IMU data 1520 describing the pronation and supination of a wearer's hand. The main board 1514 can receive other types of data describing other aspects of the pose of a wearer's hand from other types of sensors in other configurations.

The main board 1514 is connected to a host computer 1522 (participating in the collaboration session 102) via a wired or wireless connection. The host computer 1522 can be any type of computer including, but not limited to, a desktop computer, laptop computer, smartphone, tablet computer, electronic whiteboard, video game system, and augmented or virtual reality systems. The main board 1514 includes appropriate hardware to transmit sensor data to the host computer 1522. The main board 1514 of the digital glove 1500 can also receive haptic commands 1526 from the host computer 1522 instructing the digital glove 1500 to activate one or more of the haptic devices 1524.

The digital glove 1500 can be calibrated prior to use in order to provide accurate measurements for the motion and pressure of a particular wearer's hand. For instance, the digital glove 1500 might be calibrated based upon the flex of a particular wearer's hand and/or the amount of pressure exerted by the wearer. The digital glove 1500 can be constructed from cloth, leather, or another type of material.

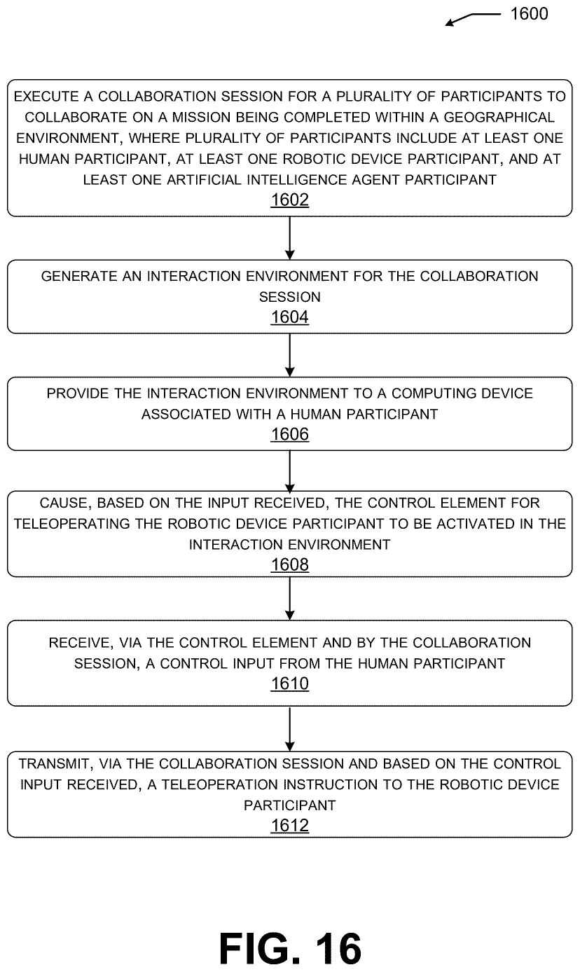

FIG. 16 is a flowchart depicting an example process 1600 for executing a collaboration session that enables a human participant to provide input that causes the collaboration session to activate (e.g., display) a control element for a robotic device participant and/or to transmit a teleoperation instruction to the robotic device participant in response to control input received via the control element.

The process 1600 begins at operation 1602 where a system executes a collaboration session for a plurality of participants to collaborate on a mission being completed within a geographical environment. As described above, the plurality of participants includes at least one human participant, at least one robotic device participant, and at last one artificial intelligence agent participant.

At operation 1604, the system generates an interaction environment for the collaboration session.

At operation 1606, the system provides the interaction environment to a computing device associated with the human participant.

At operation 1608, the system causes a control element for teleoperating the robotic device participant to be activated in the interaction environment.

At operation 1610, the system receives, via the control element and by the collaboration session, a control input from the human participant.

At operation 1612, the system transmits, via the collaboration session and based on the control input received, a teleoperation instruction to the robotic device participant.

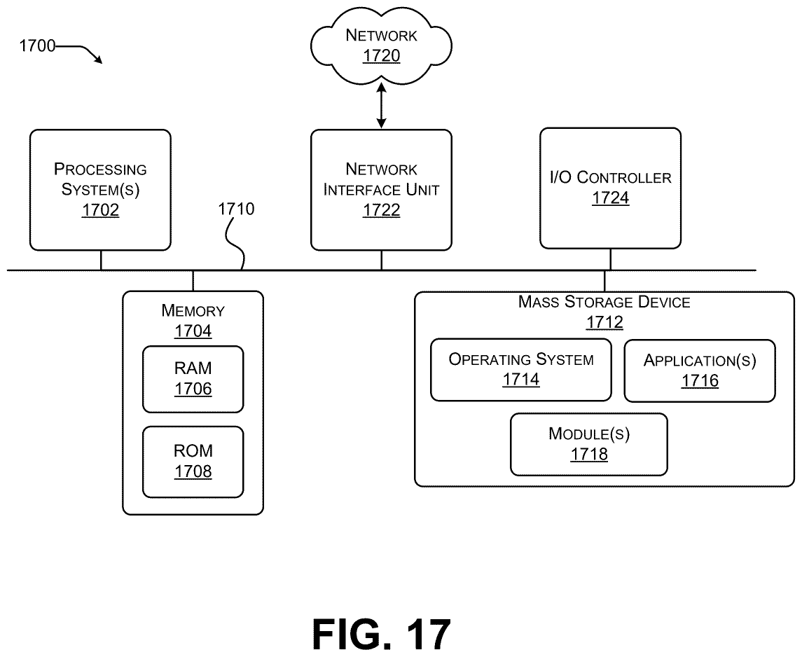

FIG. 17 shows additional details of an example computer architecture 1700 for a device, such as a computer (e.g., computing device 120) or a server configured as part of the integrated system 100, capable of executing computer instructions (e.g., a module or a program component described herein). The computer architecture 1700 illustrated in FIG. 17 includes processing unit(s) 1702, a system memory 1704, including a random-access memory 1706 (“RAM”) and a read-only memory (“ROM”) 1708, and a system bus 1710 that couples the memory 1704 to the processing unit(s) 1702.

Processing unit(s), such as processing unit(s) 1702, can represent, for example, a CPU-type processing unit, a GPU-type processing unit, a field-programmable gate array (FPGA), another class of digital signal processor (DSP), or other hardware logic components that may, in some instances, be driven by a CPU. For example, and without limitation, illustrative types of hardware logic components that can be used include Application-Specific Integrated Circuits (ASICs), Application-Specific Standard Products (ASSPs), System-on-a-Chip Systems (SOCs), Complex Programmable Logic Devices (CPLDs), etc.

A basic input/output system containing the basic routines that help to transfer information between elements within the computer architecture 1700, such as during startup, is stored in the ROM 1708. The computer architecture 1700 further includes a mass storage device 1712 for storing an operating system 1714, application(s) 1716, modules 1718, and other data described herein.

The mass storage device 1712 is connected to processing unit(s) 1702 through a mass storage controller connected to the bus 1710. The mass storage device 1712 and its associated computer-readable media provide non-volatile storage for the computer architecture 1700. Although the description of computer-readable media contained herein refers to a mass storage device, it should be appreciated by those skilled in the art that computer-readable media can be any available computer-readable storage media or communication media that can be accessed by the computer architecture 1700.

Computer-readable media can include computer-readable storage media and/or communication media. Computer-readable storage media can include one or more of volatile memory, nonvolatile memory, and/or other persistent and/or auxiliary computer storage media, removable and non-removable computer storage media implemented in any method or technology for storage of information such as computer-readable instructions, data structures, program modules, or other data. Thus, computer storage media includes tangible and/or physical forms of media included in a device and/or hardware component that is part of a device or external to a device, including but not limited to random access memory (RAM), static random-access memory (SRAM), dynamic random-access memory (DRAM), phase change memory (PCM), read-only memory (ROM), erasable programmable read-only memory (EPROM), electrically erasable programmable read-only memory (EEPROM), flash memory, compact disc read-only memory (CD-ROM), digital versatile disks (DVDs), optical cards or other optical storage media, magnetic cassettes, magnetic tape, magnetic disk storage, magnetic cards or other magnetic storage devices or media, solid-state memory devices, storage arrays, network attached storage, storage area networks, hosted computer storage or any other storage memory, storage device, and/or storage medium that can be used to store and maintain information for access by a computing device.

In contrast to computer-readable storage media, communication media can embody computer-readable instructions, data structures, program modules, or other data in a modulated data signal, such as a carrier wave, or other transmission mechanism. As defined herein, computer storage media does not include communication media. That is, computer-readable storage media does not include communications media consisting solely of a modulated data signal, a carrier wave, or a propagated signal, per se.

According to various configurations, the computer architecture 1700 may operate in a networked environment using logical connections to remote computers through the network 1720. The computer architecture 1700 may connect to the network 1720 through a network interface unit 1722 connected to the bus 1710. The computer architecture 1700 also may include an input/output controller 1724 for receiving and processing input from a number of other devices, including a keyboard, mouse, touch, or electronic stylus or pen. Similarly, the input/output controller 1724 may provide output to a display screen, a printer, or other type of output device.

It should be appreciated that the software components described herein may, when loaded into the processing unit(s) 1702 and executed, transform the processing unit(s) 1702 and the overall computer architecture 1700 from a general-purpose computing system into a special-purpose computing system customized to facilitate the functionality presented herein. The processing unit(s) 1702 may be constructed from any number of transistors or other discrete circuit elements, which may individually or collectively assume any number of states. More specifically, the processing unit(s) 1702 may operate as a finite-state machine, in response to executable instructions contained within the software modules disclosed herein. These computer-executable instructions may transform the processing unit(s) 1702 by specifying how the processing unit(s) 1702 transition between states, thereby transforming the transistors or other discrete hardware elements constituting the processing unit(s) 1702.

The disclosure presented herein also encompasses the subject matter set forth in the following clauses.

Example Clause A, a method that enables teleoperation of a robotic device by a human via a collaboration session, the method comprising: executing the collaboration session for a plurality of participants to collaborate on a mission being completed within a geographical environment, wherein the plurality of participants includes a human participant, a robotic device participant, and an artificial intelligence agent participant; generating an interaction environment for the collaboration session; providing the interaction environment to a computing device associated with the human participant; causing a control element for teleoperating the robotic device participant to be activated in the interaction environment; receiving, via the control element and by the collaboration session, a control input from the human participant; and transmitting, via the collaboration session and based on the control input received, a teleoperation instruction to the robotic device participant.

Example Clause B, the method of Example Clause A, further comprising providing, via the collaboration session, a status of a task being executed by the robotic device participant based on the teleoperation instruction transmitted to the robotic device participant.

Example Clause C, the method of Example Clause A or Example Clause B, wherein: the control element comprises a universal control element; and the teleoperation instruction causes the robotic device participant to perform a task that is common to different types of robotic device participants based on the control input received.

Example Clause D, the method of Example Clause A or Example Clause B, wherein: the control element comprises a universal control element; and the teleoperation instruction causes the robotic device participant to perform a task that is specific to a type of the robotic device participant based on the control input received.

Example Clause E, the method of Example Clause A or Example Clause B, wherein: the control element comprises a customized control element configured based on capabilities of the robotic device participant; and the teleoperation instruction causes the robotic device participant to perform a task that is specific to a type of the robotic device participant based on the control input received.

Example Clause F, the method of Example Clause A or Example Clause B, wherein the control element is configured to receive the control input from a sensory form factor device.

Example Clause G, the method of any one of Example Clauses A through F, further comprising confirming that the human participant is authorized to teleoperate the robotic device participant prior to causing the control element for teleoperating the robotic device participant to be activated in the interaction environment.

Example Clause H, the method of any one of Example Clauses A through G, further comprising: executing, within the interaction environment, a mechanism for the human participant to provide an activation input that activates the control element; and receiving, via the mechanism and by the collaboration session, the input from activation input from the human participant.

Example Clause I, the method of any one of Example Clauses A through G, wherein the control element is activated by the artificial intelligence agent participant.

Example Clause J, the method of any one of Example Clauses A through G, wherein the control element is activated in response to the robotic device participant joining the collaboration session.

Example Clause K, the method of any one of Example Clauses A through J, further comprising: receiving feedback from the robotic device participant regarding a task executed based on the teleoperation instruction; and training an artificial intelligence model that supports the artificial intelligence agent participant based on the feedback.

Example Clause L, a system comprising: a processing system; and a computer readable storage medium storing instructions that, when executed by the processing system, cause the system to perform operations comprising: executing the collaboration session for a plurality of participants to collaborate on a mission being completed within a geographical environment, wherein the plurality of participants includes a human participant, a robotic device participant, and an artificial intelligence agent participant; generating an interaction environment for the collaboration session; providing the interaction environment to a computing device associated with the human participant; causing, based on the input received, a control element for teleoperating the robotic device participant to be activated in the interaction environment; receiving, via the control element and by the collaboration session, a control input from the human participant; and transmitting, via the collaboration session and based on the control input received, a teleoperation instruction to the robotic device participant.

Example Clause M, the system of Example Clause L, wherein the operations further comprise providing, via the collaboration session, a status of a task being executed by the robotic device participant based on the teleoperation instruction transmitted to the robotic device participant.

Example Clause N, the system of Example Clause L or Example Clause M, wherein: the control element comprises a universal control element; and the teleoperation instruction causes the robotic device participant to perform a task that is common to different types of robotic device participants based on the control input received.

Example Clause O, the system of Example Clause L or Example Clause M, wherein: the control element comprises a universal control element; and the teleoperation instruction causes the robotic device participant to perform a task that is specific to a type of the robotic device participant based on the control input received.

Example Clause P, the system of Example Clause L or Example Clause M, wherein: the control element comprises a customized control element configured based on capabilities of the robotic device participant; and the teleoperation instruction causes the robotic device participant to perform a task that is specific to a type of the robotic device participant based on the control input received.

Example Clause Q, the system of Example Clause L or Example Clause M, wherein the control element is configured to receive the control input from a sensory form factor device.

Example Clause R, the system of any one of Example Clauses L through Q, wherein the operations further comprise: executing, within the interaction environment, a mechanism for the human participant to provide an activation input that activates the control element; and receiving, via the mechanism and by the collaboration session, the input from activation input from the human participant.

Example Clause S, the system of any one of Example Clauses L through Q, wherein the control element is activated by the artificial intelligence agent participant.

Example Clause T, a computer readable storage medium storing instructions that, when executed by a processing system, cause a system to perform operations comprising:
executing the collaboration session for a plurality of participants to collaborate on a mission being completed within a geographical environment, wherein the plurality of participants includes a human participant, a robotic device participant, and an artificial intelligence agent participant; generating an interaction environment for the collaboration session; providing the interaction environment to a computing device associated with the human participant; causing a control element for teleoperating the robotic device participant to be activated in the interaction environment; receiving, via the control element and by the collaboration session, a control input from the human participant; and transmitting, via the collaboration session and based on the control input received, a teleoperation instruction to the robotic device participant.

Although the various configurations have been described in language specific to structural features and/or methodological acts, it is to be understood that the subject matter defined in the appended representations is not necessarily limited to the specific features or acts described. Rather, the specific features and acts are disclosed as example forms of implementing the claimed subject matter.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without author input or prompting, whether these features, elements, and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions and changes in the form of the methods and systems described herein may be made without departing from the scope of the inventions disclosed herein. The accompanying claims and their equivalents are intended to cover such forms or modifications as would fall within the scope of certain of the inventions disclosed herein.

It should be appreciated any reference to “first,” “second,” etc. items and/or abstract concepts within the description is not intended to and should not be construed to necessarily correspond to any reference of “first,” “second,” etc. elements of the claims. In particular, within this Summary and/or the following Detailed Description, items and/or abstract concepts such as, for example, individual computing devices and/or operational states of the computing cluster may be distinguished by numerical designations without such designations corresponding to the claims or even other paragraphs of the Summary and/or Detailed Description.

In closing, although the various techniques have been described in language specific to structural features and/or methodological acts, it is to be understood that the subject matter defined in the appended representations is not necessarily limited to the specific features or acts described. Rather, the specific features and acts are disclosed as example forms of implementing the claimed subject matter.

1. A method that enables teleoperation of a robotic device by a human via a collaboration session, the method comprising:
executing the collaboration session for a plurality of participants to collaborate on a mission being completed within a geographical environment, wherein the plurality of participants includes a human participant, a robotic device participant, and an artificial intelligence agent participant;
generating an interaction environment for the collaboration session;
providing the interaction environment to a computing device associated with the human participant;
causing a control element for teleoperating the robotic device participant to be activated in the interaction environment;
receiving, via the control element and by the collaboration session, a control input from the human participant; and
transmitting, via the collaboration session and based on the control input received, a teleoperation instruction to the robotic device participant.

2. The method of claim 1, further comprising providing, via the collaboration session, a status of a task being executed by the robotic device participant based on the teleoperation instruction transmitted to the robotic device participant.

3. The method of claim 1, wherein:
the control element comprises a universal control element; and
the teleoperation instruction causes the robotic device participant to perform a task that is common to different types of robotic device participants based on the control input received.

4. The method of claim 1, wherein:
the control element comprises a universal control element; and
the teleoperation instruction causes the robotic device participant to perform a task that is specific to a type of the robotic device participant based on the control input received.

5. The method of claim 1, wherein:
the control element comprises a customized control element configured based on capabilities of the robotic device participant; and
the teleoperation instruction causes the robotic device participant to perform a task that is specific to a type of the robotic device participant based on the control input received.

6. The method of claim 1, wherein the control element is configured to receive the control input from a sensory form factor device.

7. The method of claim 1, further comprising confirming that the human participant is authorized to teleoperate the robotic device participant prior to causing the control element for teleoperating the robotic device participant to be activated in the interaction environment.

8. The method of claim 1, further comprising:
executing, within the interaction environment, a mechanism for the human participant to provide an activation input that activates the control element; and
receiving, via the mechanism and by the collaboration session, the input from activation input from the human participant.

9. The method of claim 1, wherein the control element is activated by the artificial intelligence agent participant.

10. The method of claim 1, wherein the control element is activated in response to the robotic device participant joining the collaboration session.

11. The method of claim 1, further comprising:
receiving feedback from the robotic device participant regarding a task executed based on the teleoperation instruction; and
training an artificial intelligence model that supports the artificial intelligence agent participant based on the feedback.

12. A system comprising:
a processing system; and
a computer readable storage medium storing instructions that, when executed by the processing system, cause the system to perform operations comprising:
executing the collaboration session for a plurality of participants to collaborate on a mission being completed within a geographical environment, wherein the plurality of participants includes a human participant, a robotic device participant, and an artificial intelligence agent participant;
generating an interaction environment for the collaboration session;
providing the interaction environment to a computing device associated with the human participant;
causing, based on the input received, a control element for teleoperating the robotic device participant to be activated in the interaction environment;
receiving, via the control element and by the collaboration session, a control input from the human participant; and
transmitting, via the collaboration session and based on the control input received, a teleoperation instruction to the robotic device participant.

13. The system of claim 12, wherein the operations further comprise providing, via the collaboration session, a status of a task being executed by the robotic device participant based on the teleoperation instruction transmitted to the robotic device participant.

14. The system of claim 12, wherein:
the control element comprises a universal control element; and
the teleoperation instruction causes the robotic device participant to perform a task that is common to different types of robotic device participants based on the control input received.

15. The system of claim 12, wherein:
the control element comprises a universal control element; and
the teleoperation instruction causes the robotic device participant to perform a task that is specific to a type of the robotic device participant based on the control input received.

16. The system of claim 12, wherein:
the control element comprises a customized control element configured based on capabilities of the robotic device participant; and
the teleoperation instruction causes the robotic device participant to perform a task that is specific to a type of the robotic device participant based on the control input received.

17. The system of claim 12, wherein the control element is configured to receive the control input from a sensory form factor device.

18. The system of claim 12, wherein the operations further comprise:
executing, within the interaction environment, a mechanism for the human participant to provide an activation input that activates the control element; and
receiving, via the mechanism and by the collaboration session, the input from activation input from the human participant.

19. The system of claim 12, wherein the control element is activated by the artificial intelligence agent participant.

20. A computer readable storage medium storing instructions that, when executed by a processing system, cause a system to perform operations comprising:
executing the collaboration session for a plurality of participants to collaborate on a mission being completed within a geographical environment, wherein the plurality of participants includes a human participant, a robotic device participant, and an artificial intelligence agent participant;
generating an interaction environment for the collaboration session;
providing the interaction environment to a computing device associated with the human participant;
causing a control element for teleoperating the robotic device participant to be activated in the interaction environment;
receiving, via the control element and by the collaboration session, a control input from the human participant; and
transmitting, via the collaboration session and based on the control input received, a teleoperation instruction to the robotic device participant.
